SEQUENCE LISTING (6 sequences)

SEQ ID NO: 1 - protein, 2386 aa, homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 2 - DNA, 20 bp, homo sapiens
tcttagcacctggcgtttca

SEQ ID NO: 3 - DNA, 20 bp, homo sapiens
cgaccctctgtttccaggag

SEQ ID NO: 4 - DNA, 21 bp, homo sapiens
cacctgctcggtgacatatta

SEQ ID NO: 5 - RNA, 19 nt, homo sapiens
ggaaacuaugucugcgaaa

SEQ ID NO: 6 - RNA, 19 nt, homo sapiens
uucuccgaacgugucacgu

(19) United States
(12) Patent Application Publication
(10) Pub. No.: US 2016/0067314 A1
(43) Pub. Date: Mar. 10, 2016

(54) COMPOSITIONS AND METHODS FOR INHIBITING CHEMORESISTANCE IN CANCER AND IMPROVING RESPONSE TO THERAPY

(71) Applicants: Martin A. SCHWARTZ, (US); Kumari P. DARAWEWA, Charlottesville, VA (US); Konstadinos MOISSOGLU, Charlottesville, VA (US); UNIVERSITY OF VIRGINIA PATENT FOUNDATION, Charlottesville, VA (US)

(72) Inventors: Martin A. SCHWARTZ, Milford, CT (US); P. Kumari ANDARAWEWA, Charlottesville, VA (US); Konstadinos MOISSOGLU, Charlottesville, VA (US)

(73) Assignee: University of Virginia Patent Foundation, Charlottesville, VA (US)

(21) Appl. No.: 14/785,588

(22) PCT Filed: Apr. 18, 2014
(86) PCT No.: PCT/US14/34648

§ 371 Date: Oct. 19, 2015

Related U.S. Application Data
(60) Provisional application No. 61/813,866, filed on Apr. 19, 2013.
(60) Provisional application No. 61/813,896, filed on Apr. 19, 2013.

(51) Int. Cl. A61K 38/39; C07K 14/78; A61K 31/713; C07K 16/28; C12Q 1/68; G01N 33/574; C07K 16/30; A61K 39/395; A61K 45/06

(52) U.S. Cl. CPC A61K 38/39; A61K 39/39558; C07K 14/78; A61K 31/713; A61K 45/06; C12Q 1/6886; G01N 33/5743; C07K 16/3053; C07K 16/2803; A61K 2039/505

(57) ABSTRACT

Metastatic melanomas are highly resistant to radiation and chemotherapy from the earliest stages, which is a major factor in poor clinical outcomes. Activated leukocyte adhesion molecule (ALCAM)/CD166 was the gene that showed the highest correlation with detachment-induced chemoresistance. SiRNA-mediated depletion or antibody blocking of ALCAM specifically inhibited the increase in chemoresistance after detachment. This antibody also improved chemotherapeutic responses in a mouse xenograft model of human melanoma. Previous studies identified ALCAM as a marker for tumor aggressiveness and poor prognosis, and as a marker for “stemness”. Targeting ALCAM may therefore represent a novel approach for treatment of otherwise intractable melanomas. It was also found that stimulating integrin signaling enhanced chemosensitivity of melanoma to chemotherapeutic agents. The present invention provides a novel multimeric peptide construct comprising fibronectin fragments useful for stimulating integrin signaling and for enhancing chemosensitivity of melanomas.

### CROSS REFERENCE TO RELATED APPLICATIONS

This application is entitled to priority pursuant to 35 U.S.C. §119(e) to U.S. provisional patent application No. 61/813,866, filed on Sep. Apr. 19, 2013 and to U.S. provisional patent application No. 61/813,896, filed on Sep. Apr. 19, 2013. The entire disclosures of the afore-mentioned patent applications are incorporated herein by reference.

### BACKGROUND

Metastatic melanomas are highly lethal due to their rapid spread and early acquisition of resistance to chemotherapy and radiation. However, the reasons for therapy resistance are not well understood. Many melanomas have wild-type p53 genes and lack known defects in other DNA damage and apoptosis pathways. Recent work has suggested instead that microenvironmental interactions are an important factor in melanoma therapy resistance (Schwartz, 2008). The majority of melanomas cells within tumors reside in “nests” that are depleted of extracellular matrix (ECM). For most melanomas examined in vitro, adhesion to ECM proteins increases sensitivity to chemotherapy (Schwartz, 2008). These effects are mediated by synergies between integrins and both p53- and p73-dependent DNA damage pathways (Lewis, 2002; Truong, 2003). Mouse embryo fibroblasts and human fibrosarcoma also exhibit this behavior, whereas a number of carcinomas exhibit the opposite behavior, that integrin-mediated adhesion enhances survival and decreases chemo- and radiation sensitivity (Hodkinson, 2007, Int. J. Radiat. Biol. 83:733-41). Moreover, melanomas isolated from different patients show large variations in the adhesion-dependence of therapeutic responsiveness. There is variability in integrin dependence of chemosensitivity.

Chemotherapy can be ineffective for treating melanoma. For non-resectable tumors, five-year survival is less than 10%. Methods that would help identify susceptible tumors or induce chemoresistant tumor to become chemosensitive could revolutionize treatment of melanoma.

There is a long felt need in the art for compositions and methods useful for overcoming chemoresistance and treating cancer. The present invention satisfies these needs.

### SUMMARY OF THE INVENTION

Previous work showed that many but not all melanomas are inherently sensitive to chemotherapy but become resistant as a consequence of changes in interactions with surrounding extracellular matrix (ECM). These tumors become resistant to chemotherapy when the ECM within the tumor is degraded and integrins are not engaged and signaling.

In the Examples disclosed herein, data are provided from studies investigating the molecular basis for the variability in integrin dependence of chemosensitivity to chemotherapeutic agents in melanoma. DNA microarray analysis of gene expression in melanoma lines showed that activated leukocyte adhesion molecule (ALCAM)/CD166 correlates with detachment-induced chemoresistance, that expression of this protein was required for chemoresistance in non-adherent cells, and that inhibiting ALCAM levels or activity reduced chemoresistance. The present invention therefore encompasses the use of antibodies and other antagonists of ALCAM for treatment of patients with metastatic melanoma and further encompasses the combination of these antagonists with other agents.

Previous work showed that activating integrin signaling improved chemosensitivity in most melanomas, however, the treatments used in those studies are not suitable for use in human patients. Disclosed herein is a pentameric (i.e., multimeric) fibronectin construct, FN-COMP, was designed for this purpose. It is disclosed herein that FN-COMP activated integrin signaling, increased melanoma chemosensitivity and radiosensitivity in vitro, and significantly improved responses to chemotherapy in vivo. FN-COMP may therefore be useful for treatment of metastatic melanoma in humans. FN-COMP is a homo-pentameric construct of fibronectin peptides linked by the assembly domain of COMP (amino acid residues 27-84 of COMP) and each fibronectin peptides comprises type III repeats 5-11 of fibronectin.

Fibronectin has the amino acid sequence provided herein (SEQ ID NO:1; NCBI/GenBank accession number P02751, comprising 2386 aa). Use of the term “fibronectin peptide” refers to fibronectin or a fragment thereof. The fibronectin type III domain repeat region is an approximately 100 amino acid domain, different tandem repeats of which contain binding sites for DNA, heparin and the cell surface. The superfamily of sequences believed to contain FnIII repeats represents 45 different families, the majority of which are involved in cell surface binding in some manner, or are receptor protein tyrosine kinases, or cytokine receptors.

The type III domain regions of fibronectin can be found between residue numbers 610 and 702 (1), 722 and 812 (2), 813 and 904 (3), 909 and 998 (4), 999 and 1098 (5), 1089 and 1175 (6), 1176 and 1266 (7), 1269 and 1361 (8), 1362 and 1449 (9), 1450 and 1543 (10), 1544 and 1635 (11), 1636 and 1723 (12), 1724 and 1817 (13), 1818 and 1904 (14), 1905 and 1995 (15), 2103 and 2197 (16), respectively. The regions “underlined” relate to type III repeats 5-11 of fibronectin, which are used in preparing FN-COMP. It is disclosed that this reagent stimulates integrin signaling and improves chemosensitivity in vitro and in vivo. In one aspect, FN-COMP sensitizes melanoma cells to therapy in vivo, improving the outcome. In one aspect, FN-COMP is useful for inhibiting tumor growth.

One of ordinary skill in the art will appreciate that the sequences can be modified with conservative amino acid changes, including, insertions, deletions, and substitutions, and that the valency could be altered as well, as long as the resulting multimer/multimeric complex remains effective. Amino acid changes (fragments and homologs) can be made independently in each fibronectin and in COMP. One of ordinary skill in the art will appreciate that C-terminal groups other than His6 can be used. One of skill in the art will also realize that the pentameric, or other valency, complex can be linked in different ways.

For cancers where integrin-mediated adhesion promotes cell death and growth arrest in response to DNA damage, this treatment will lead to improved therapeutic responses.

The present invention provides for a combination therapy using a multimeric complex of the invention and a chemotherapeutic agent or other therapy, such as radiotherapy or immunotherapy. In one aspect, the agent is a drug. In one aspect, the compositions and methods are useful for treating cancer. In one aspect, the compositions and methods are useful for treating metastatic cancer. In another aspect, the compositions and methods are useful for treating any cancer where integrin signaling is involved in the cancer growth. In one aspect, the cancer is melanoma.

Useful agents for treating melanoma include, but are not limited to, Dacarbazine (also called DTIC), Temozolomide, Taxanes, Nab-paclitaxel, Paclitaxel, Nitrosureas, Carmustine (also known as BCNU), Platinum-based agents, Cisplatin, Carboplatin, Vinblastine, Interferon-alpha, Interleukin-2, Ipilimumab, and Vemurafenib.

Platinum-based chemotherapeutic agents useful in the practice of the invention, include, but are not limited to, cisplatin, carboplatin, oxaliplatin, satraplatin, picoplatin, nedaplatin, triplatin, and lipoplatin. In one embodiment, carboplatin is administered intravenously for a time period of at least about 5 minutes. One of ordinary skill in the art will appreciate that the dosage of carboplatin or other platinum-based chemotherapeutic agents is based on health, weight, body size, and response to therapy and that these can be used in conjunction with a known dose calculator. For example, some recent guidelines suggest that the maximum dose is based on a glomerular filtration rate (GFR) estimate that is capped at 125 mL/min for patients with normal renal function and that no higher estimated GFR values should be used.

In general, courses of cisplatin treatment are not given more often than once every four weeks, but can be varied. It can be administered, for example, about once a week, once every two weeks, once every three weeks, once every four weeks, once every five weeks, once every six weeks, once every eight weeks and once every ten weeks. The aforementioned timing regimen is not meant to be exhaustive or exclusive and can include administering the agent at least twice per each time frame, etc.

One of ordinary skill in the art will appreciate that other fragments of fibronectin can be used with the present invention wherein said fragments comprise at least one type III repeat domain and bind with at least one integrin on the target melanoma cells.

In one embodiment, the present invention provides a method for enhancing sensitivity to chemotherapeutic agents in melanoma cells that are resistant to the chemotherapeutic agent before being sensitized. In one aspect, once sensitized, the cells are then contacted with a chemotherapeutic agent. The method comprises contacting the cells with an effective amount of at least one integrin signaling stimulating agent and optionally at least one inhibitor of ALCAM levels or activity to enhance sensitivity of the melanoma cells to the chemotherapeutic agent. The treatment is more effective after the cells have been sensitized than without sensitization.

In one embodiment of enhancing sensitivity, the integrin signaling stimulating agent is a multimeric fibronectin construct comprising at least two fibronectin peptides and a linker. In one aspect, fibronectin has SEQ ID NO:1, or a homolog or fragment thereof. In one aspect, the multimeric fibronectin construct comprises as the linker short cartilage oligomeric peptide (COMP) or a homolog or fragment thereof. In one aspect, the multimeric fibronectin construct comprises five fibronectin peptides linked by COMP or a homolog or fragment thereof. In another aspect, the fibronectin peptides comprises at least one type III repeat domain of fibronectin. In another aspect, each of the fibronectin peptides comprises at least two type III repeats. In yet another aspect, each of the fibronectin peptides comprises type III repeats 5, 6, 7, 8, 9, 10, and 11. In one aspect, the COMP comprises the assembly domain of COMP. In one aspect, the assembly domain comprises amino acid residues 27-84 of COMP.

In one embodiment of the construct, the linker comprises amino acid residues 27-84 of COMP, the construct is a homo-multimeric construct comprising five fibronectin peptides, and each of the fibronectin peptides comprises type III repeats 5, 6, 7, 8, 9, 10, and 11 of fibronectin (FN-COMP).

In one aspect, the integrin signaling agent binds to at least one integrin on the melanoma cell. In one aspect, the integrins are selected from the group consisting of α5β1, αVβ3, and α4β1.

In one aspect, of the sensitization, the cell is contacted with an effective amount of an inhibitor of ALCAM levels or activity.

In one aspect, the integrin signaling stimulating agent is FN-COMP.

In one aspect, the inhibitor of ALCAM is an siRNA directed against ALCAM. In a further aspect, the siRNA has the sequence of SEQ ID NO:4 or SEQ ID NO:5.

In one aspect, the inhibitor of ALCAM levels or activity is an antibody directed against ALCAM. In one aspect, an antibody of the invention includes, but is not limited to, monoclonal antibodies, single chain antibodies, synthetic antibodies, humanized antibodies, and chimeric antibodies, and biologically active fragments and homologs thereof. In one aspect, the antibody is a monoclonal antibody. In one aspect, the monoclonal antibody is AZN-L50.

In one aspect, an antibody of the invention can be administered to a subject at a dosage from about 0.01 mg/kg to about 100 mg/kg, about 0.1 mg/kg to about 75 mg/kg, about 0.5 mg/kg to about 50 mg/kg, about 1.0 mg/kg to about 25 mg/kg, about 2.0 mg/kg to about 20 mg/kg, about 3.0 mg/kg to about 15 mg/kg, about 4.0 mg/kg to about 10 mg/kg, or about 5.0 mg/kg to about 7.5 mg/kg. The present invention further encompasses the administration of unit doses, which can be, for example, 1, 5, 10, 20, 25, 30, 35, 40, 45, 50, 55, 60, 65, 70, 75, 80, 85, 90, 95, 100, 125, 150, 200, 500, 1,000, or 5,000 mg.

Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

In one aspect, the cells are sensitized and then contacted with a chemotherapeutic agent. One of ordinary skill in the art will appreciated that depending on whether the melanoma is chemoresistant or not, the timing and sequence of administration of at least one antibody directed against ALCAM or at least on integrin signaling stimulating agent relative to administration of a chemotherapeutic agent can be varied.

In one aspect, the chemotherapeutic agent is dacarbazine, temozolomide, taxanes, nab-paclitaxel, paclitaxel, nitrosureas, carmustine, platinum-based agents, cisplatin, carboplatin, oxaliplatin, satraplatin, picoplatin, nedaplatin, triplatin, lipoplatin, or vinblastine.

In one embodiment, the present invention provides method for determining whether a melanoma is a detachment-induced therapy resistant melanoma. The method comprises measuring the level of ALCAM in the melanoma, comparing the level of ALCAM with the level of ALCAM in a sample from a second melanoma that is not detachment-induced therapy resistant or comparing the level with a standard sample comprising a known level of ALCAM, wherein a higher level of ALCAM in the melanoma compared to the second melanoma or the standard is an indication that the melanoma is detachment-induced therapy resistant. In one aspect, a high level of ALCAM is an indication that the melanoma will be responsive to a treatment comprising integrin signaling stimulation in conjunction with chemotherapy or to treatment to reduce ALCAM levels or expression, or both. In one embodiment, a treatment regimen is designed based on the ALCAM level of the melanoma. In one aspect, the treatment comprises administering to a subject in need thereof a pharmaceutical composition comprising an effective amount of a chemotherapeutic agent and either an effective amount of at least one integrin signaling stimulating agent or an effective amount of at least one inhibitor of ALCAM levels or activity, or both, and optionally an additional therapeutic agent. In one aspect, the ALCAM level measured is ALCAM protein level, ALCAM mRNA level, or both protein and mRNA levels.

In one embodiment, the present invention comprises obtaining a melanoma sample from a patient and then measuring the ALCAM levels in the sample and determining that the melanoma is chemotherapy resistant when the ALCAM levels are high. In one aspect, the information is used to develop treatment regimens.

The present invention encompasses compositions and methods useful for blocking or inhibiting (ALCAM)/CD166 function, expression, levels and synthesis to overcome its role in chemoresistance of melanomas. In one aspect, (ALCAM)/CD166 is inhibited using an antibody directed against (ALCAM)/CD166. In one aspect, the antibody is a monoclonal antibody. In one aspect, the monoclonal antibody is AZN-L50. In one aspect, ALCAM is inhibited using siRNA directed against ALCAM. In one aspect, the siRNA is siRNA #11 (SEQ ID NO:4) or siRNA #10 (SEQ ID NO:5) One of ordinary skill in the art will appreciate that other types of molecules that can inhibit ALCAM expression, synthesis, levels, and activity are encompassed by the invention, including antisense oligonucleotides and aptamers.

In one embodiment, ALCAM serves as a marker for detachment-induced therapy resistance in melanoma. In one aspect, the resistance is to chemotherapy. In one aspect, the compositions and methods of the invention are useful for improving chemotherapeutic response in melanomas. In one aspect, they are useful for improving the response to other therapies, including, but not limited to, immunotherapy and radiotherapy.

The present invention further provides a method for differentiating a detachment-induced therapy resistant melanoma from a therapy sensitive melanoma. The method comprises measuring the level of ALCAM in a test melanoma, comparing the level of ALCAM with the level of ALCAM in a sample from a second melanoma that is not detachment-induced therapy resistant or with a standard sample comprising a known level of ALCAM, wherein a higher level of ALCAM in the test melanoma compared to the second melanoma or the standard is an indication that the melanoma is detachment-induced therapy resistant. One of ordinary skill in the art can determine what standard to use.

In one embodiment, the present invention provides a multimeric fibronectin construct for stimulating integrin signaling, said construct comprising at least two fibronectin peptides and a linker. In one aspect, the multimer is a homo-multimer of fibronectin peptides. In one aspect, the construct comprises five fibronectin peptides. In another aspect, the construct is FN-COMP. In a further aspect, the multimeric fibronectin construct is a homo-multimer or heteromultimer of fibronectin peptides or homologs or fragments thereof. In one aspect, the multimeric fibronectin construct comprises the linker COMP or a fragment thereof. In one aspect, the multimeric fibronectin construct comprises five fibronectin peptides linked by COMP or a homolog or fragment thereof. In one aspect, the fibronectin peptides comprises at least one type III domain repeat of fibronectin. There are three types of internal repeat with fibronectin. In another aspect, each of the fibronectin peptides comprises at least two type III repeats. In a further aspect, each of said fibronectin peptides comprises type III repeats 5, 6, 7, 8, 9, 10, and 11. In another aspect, the COMP fragment comprises the assembly domain of COMP. In one aspect, the assembly domain comprises amino acid residues 27-84 of COMP. In one aspect, the linker comprises amino acid residues 27-84 of COMP, the construct is a homo-multimeric construct comprising five fibronectin peptides (a pentamer), and each of the five fibronectin peptides comprises type III repeats 5, 6, 7, 8, 9, 10, and 11 of fibronectin (FN-COMP). In one aspect, the construct binds to at least one integrin on a melanoma cell. In one aspect, the integrin is selected from the group consisting of α5β1, αVβ3, and α4β1.

In one embodiment, the invention provides a method for treating a melanoma having detachment induced-chemoresistance wherein the method enhances sensitivity of the melanoma to a chemotherapeutic agent. The method comprises administering to a subject in need thereof a pharmaceutical composition comprising an effective amount of a chemotherapeutic agent and either an effective amount of at least one integrin signaling stimulating agent or an effective amount of at least one inhibitor of ALCAM levels or activity, or both, and optionally an additional therapeutic agent. In one aspect, the integrin signaling stimulating agent is a multimeric fibronectin construct comprising at least two fibronectin peptides and a linker.

In one aspect, the multimeric fibronectin construct comprises the linker short cartilage oligomeric peptide (COMP) or a fragment thereof. In one aspect, the multimeric fibronectin construct comprises five fibronectin peptides, or homologs or fragment thereof, linked by COMP. In one aspect, the fibronectin of a construct of the invention has the sequence of SEQ ID NO:1, or fragments or homologs thereof. In one aspect, each of the fibronectin peptides comprises at least one type III repeat of fibronectin. In one aspect, each of the fibronectin peptides comprises at least two type III repeats. In another aspect, each of the fibronectin peptides comprises type III repeats 5, 6, 7, 8, 9, 10, and 11. In one aspect, the COMP comprises the assembly domain of COMP. In another aspect, the assembly domain comprises amino acid residues 27-84 of COMP. In one aspect, the linker comprises amino acid residues 27-84 of COMP, the construct is a homo-multimeric construct comprising five fibronectin peptides (pentamer), and each of the fibronectin peptides comprises type III repeats 5, 6, 7, 8, 9, 10, and 11 of fibronectin (FN-COMP).

In one aspect, the integrin signaling agent binds to at least one integrin on a melanoma cell of the melanoma. In one aspect, the integrin is selected from the group consisting of α5β1, αVβ3, and α4β1. In one aspect, an effective amount of an inhibitor of ALCAM levels or activity is administered to the subject. In one aspect, an effective amount of at least one integrin signaling stimulating agent and an effective amount of at least one inhibitor of ALCAM levels or activity are administered to the subject. In one aspect, prior to treatment it is determined whether the melanoma is a detachment-induced therapy resistant melanoma and a treatment is designed based on the outcome of the determination.

In one embodiment, a multimeric peptide construct dosage of about 0.1 mg/kg to about 100 mg/kg can be administered to a subject in need thereof, including whole numbers between 0.1 and 100 and fractions thereof. In one aspect, a multimeric peptide construct dosage of about 1.0 mg/kg to about 75 mg/kg can be administered to a subject. In another aspect, a multimeric peptide construct dosage of about 5.0 mg/kg to about 50 mg/kg can be administered to a subject. In yet another aspect, a multimeric peptide construct dosage of about 10 mg/kg to about 25 mg/kg can be administered to a subject. In a further aspect, a multimeric peptide construct dosage of about 15 mg/kg to about 20 mg/kg can be administered to a subject. In one aspect, the multimeric peptide construct is a fibronectin peptide construct. In one aspect, the fibronectin peptide construct is FN-COMP. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

In one embodiment, a unit dose of multimeric peptide construct can be administered. Other therapeutic agents of the invention can also be administered as unit doses. Unit doses include, but are not limited to, 0.1 mg, 1.0 mg, 5.0 mg, 10 mg, 25, mg, 50, mg, 100 mg, 150 mg, 200 mg, 500 mg, 1,000 mg, 1,500 mg, 5,000 mg, and 10,000 mg. Kits can be provided with unit doses in a container or syringe or amounts that one of ordinary skill in the art can administer based on a dose per weight, etc.

In one embodiment, a multimeric peptide construct of the invention is administered at least once a day, or once a week, or once month. In one embodiment, a multimeric peptide construct of the invention is administered at least twice a day, or twice a week, or twice a month.

In one aspect, the present invention provides compositions and methods for determining whether a melanoma is a detachment-induced therapy resistant melanoma by measuring the level of ALCAM in the melanoma, comparing the level of ALCAM with the level of ALCAM in a sample from a second melanoma that is not detachment-induced therapy resistant or with a standard sample comprising a known level of ALCAM, wherein a higher level of ALCAM in the melanoma compared to the second melanoma or said standard is an indication that the melanoma is detachment-induced therapy resistant.

In one embodiment, the present invention provides a method for treating chemoresistant melanoma in a subject in need thereof, wherein the chemoresistance is associated with high ALCAM levels in the melanoma. The method comprises administering to the subject a pharmaceutical composition comprising a pharmaceutically acceptable carrier and an effective of amount of at least one chemotherapeutic agent, an effective amount of at least one integrin signaling stimulator, optionally at least one inhibitor of ALCAM levels or activity, and optionally an additional therapeutic agent.

In one embodiment, the present invention provides compositions and methods for establishing a treatment regimen for a subject with melanoma. The method comprises determining the level of ALCAM expression in the melanoma, comparing the level of ALCAM with the level of ALCAM in a sample from a second melanoma that is not detachment-induced therapy resistant or with a standard sample comprising a known level of ALCAM. In one aspect, if it is determined that there is a higher level of ALCAM in the test melanoma compared to the second melanoma or the standard it is an indication that the melanoma is detachment-induced therapy resistant, thereby differentiating a detachment-induced therapy resistant melanoma from a therapy sensitive melanoma. Then, a treatment plan for the subject should include administering a pharmaceutical composition comprising an effective amount of at least one chemotherapeutic or other therapeutic agent, an effective amount of at least one integrin stimulating agent, and optionally at least one inhibitor of ALCAM levels or activity. However, in another aspect, if it is determined that there is a similar level of ALCAM in the test melanoma compared to the second melanoma that is not detachment-induced therapy resistant or with a standard sample comprising a known level of ALCAM, it is an indication that the test melanoma is not detachment-induced therapy resistant and that a treatment plan for the subject should comprise administering a pharmaceutical composition comprising an effective amount of a standard melanoma chemotherapeutic agent or other therapy or agent. In one aspect, the method is useful for predicting responsiveness to chemotherapy. That is, if high ALCAM levels are found in a melanoma, the melanoma is chemotherapy resistant and the invention encompasses designing a treatment regimen coupled with the determination of whether the melanoma is found to be chemotherapy resistant or not. Additionally, when it is determined that the melanoma is chemoresistant, the predicted successful treatment will include the use of agents to inhibit ALCAM expression levels and/or the use of agents to stimulate integrin signaling.

The compositions of the invention are also useful for monitoring the progression and treatment of a melanoma in a subject. In one aspect, a sample can be obtained from a subject and ALCAM levels can be determined. In another aspect, ALCAM can be detected in vivo using imaging techniques, such as administering an anti-ALCAM antibody coupled with an imaging agent.

Any reference to a series of types of subtypes of a molecule, or dose, or treatment is not intended to be limited to just the specific embodiment in which it is described.

### Sequences of the Invention

SEQ ID NO: 1 (fibronectin-GenBank accession number
P02751):
mlrgpgpgllllavqclgtavpstgaskskrqaqqmvqpqspvaysqskp
gcydngkhyqinqqwertylgnalvctcyggsrgfnceskpeaeetcfdk
ytgntyrvgdtyerpkdsmiwdctcigagrgrisctianrcheggqsyki
gdtwaphetggymlecyclgngkgewtckpiaekcfdhaagtsyvvgetw
ekpyqgwmmvdctclgegsgritctsrnrcndqdtrtsyrigdtwskkdn
rgnllqcictgngrgewkcerhtsvqttssgsgpftdvraavyqpqphpq
pppyghcvtdsgvvysvgmqwlktqgnkqmlctclgngvscqetavtqty
ggnsngepcvlpftyngrtfyscttegrqdghlwcsttsnyeqdqkysfc
tdhtvlvqtrggnsngalchfpflynnhnytdctsegrrdnmkwcgttqn
ydadqkfgfcpmaaheeicttnegymyrigdqwdkqhdmghmmrctcvgn
grgewtciaysqlrdqcivdditynvndtfhkrheeghmlnctcfgqgrg
rwkcdpvdqcqdsetgtfyqigdswekyvhgvryqcycygrgigewhcqp
lqtypsssgpvevfitetpsqpnshpiqwnapqpshiskyilrwrpknsv
grwkeatipghlnsytikglkpgvvyegqlisiqqyghqevtrfdfttts
tstpvtsntvtgettpfsplvatsesvteitassfvvswvsasdtvsgfr
veyelseegdepqyldlpstatsvnipdllpgrkyivnvyqisedgeqsl
ilstsqttapdappdttvdqvddtsivvrwsrpqapitgyrivyspsveg
sstelnlpetansvtlsdlqpgvqynitiyaveenqestpvviqqettgt
prsdtvpsprdlqfvevtdvkvtimwtppesavtgyrvdvipvnlpgehg
qrlpisrntfaevtglspgvtyyfkvfayshgreskpltaqqttkldapt
nlqfvnetdstvlvrwtppraqitgyrltvgltrrgqprqynvgpsysky
plrnlqpaseytvslvaikgnqespkatgvfttlqpgssippyntevtet
tivitwtpaprigfklgvrpsqggeaprevtsdsgsivvsgltpgveyvy
tiqvirdgqerdapivnkvvtplspptnlhleanpdtgvltvswersttp
ditgyritttptngqqgnsleevvhadqssctfdnlspgleynvsvytvk
ddkesvpisdtiipavppptdlrftnigpdtmrvtwapppsidltnflvr
yspvkneedvaelsispsdnavvlinllpgteyvvsyssvyeqhestplr
grqktgldsptgidfsditansftvhwiapratitgyrirhhpehfsgrp
redrvphsrnsitltnitpgteyvvsivalngreesplligqqstvsdvp
rdlevvaatptslliswdapavtvryyritygetggnspvqeftvpgsks
tatisglkpgvdytitvyavtgrgdspasskpisinyrteidkpsqmqvt
dvqdnsisvkwlpssspvtgyrytttpkngpgptktktagpdqtemtieg
lqptveyvvsvyaqnpsgesqplvqtavtnidrpkglaftdvdvdsikia
wespqgqvsryrytysspedgihelfpapdgeedtaelqglrpgseytvs
vvalhddmesqpligtqstaipaptdlkftqvtptslsaqwtppnvqltg
yrvrvtpkektgpmkeinlapdsssvvvsglmvatkyevsvyalkdtlts
rpaqgvvttlenvspprrarvtdatettitiswrtktetitgfqvdavpa
ngqtpiqrtikpdvrsytitglqpgtdykiylytlndnarsspvvidast
aidapsnlrflattpnsllvswqppraritgyiikyekpgspprevvprp
rpgvteatitglepgteytiyvialknnqksepligrkktdelpqlvtlp
hpnlhgpeildvpstvqktpfvthpgydtgngiqlpgtsgqqpsvgqqmi
feehgfrrttppttatpirhrprpyppnvgeeigighipredvdyhlyph
gpglnpnastgqealsqttiswapfqdtseyiischpvgtdeeplqfrvp
gtstsatltgltrgatynvivealkdqqrhkvreevvtvgnsvneglnqp
tddscfdpytvshyavgdewermsesgfkllcqclgfgsghfrcdssrwc
hdngvnykigekwdrqgengqmmsctclgngkgefkcdpheatcyddgkt
yhvgeqwqkeylgaicsctcfggqrgwrcdncrrpggepspegttgqsyn
qysqryhqrtntnvncpiecfmpldvqadredsre
SEQ ID NO: 2-ALCAM forward primer:
5-TCTTAGCACCTGGCGTTTCA-3
SEQ ID NO: 3-ALCAM reverse primer:
5-CGACCCTCTGTTTCCAGGAG-3
SEQ ID NO: 4-ALCAM siRNA #11:
5′-CACCTGCTCGGTGACATATTA-3′
SEQ ID NO: 5-ALCAM siRNA #10:
5-GGAAACUAUGUCUGCGAAA-3
SEQ ID NO: 6-ALCAM control siRNA:
UUCUCCGAACGUGUCACGU

Various aspects and embodiments of the invention are described in further detail below.

### BRIEF DESCRIPTION OF THE DRAWINGS

### Example 1

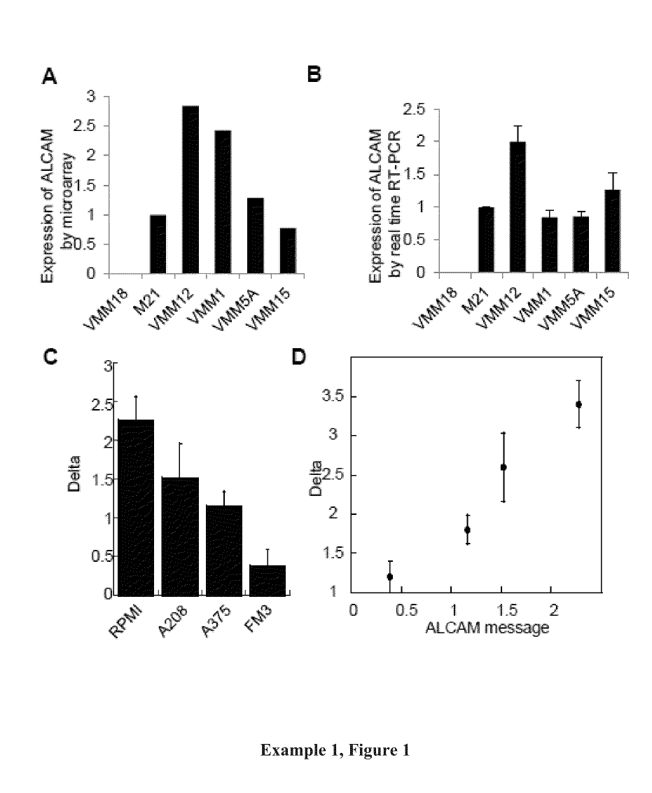

FIG. 1. ALCAM correlates with detachment-induced therapy resistance. Relative ALCAM expression levels in melanoma measured by (A) microarray analysis and (B) RT-PCR. Expression levels are normalized to M21. Values are means±SE. (C) Relative ALCAM expression versus cell survival in additional melanoma cell lines. The graph represents detachment-induced resistance (“delta”) as the fold difference in survival between suspended versus adherent cells. Survival of adherent and suspended cells after treatment with chemotherapy (Ara-C) for 3 days was determined by replating on fibronectin. Concentrations of Ara-C were chosen for each cell line to kill ˜85% of adherent cells as determined in preliminary experiments. Concentrations were: A208 cells, 0.5 μM; A375.S2 cells, 0.3 μM; RPMI7951 cells, 5 μM, FM3: 10 μM). (D) Delta was plotted against ALCAM message level determined by RT-PCR. Values are means±SE, n>3.

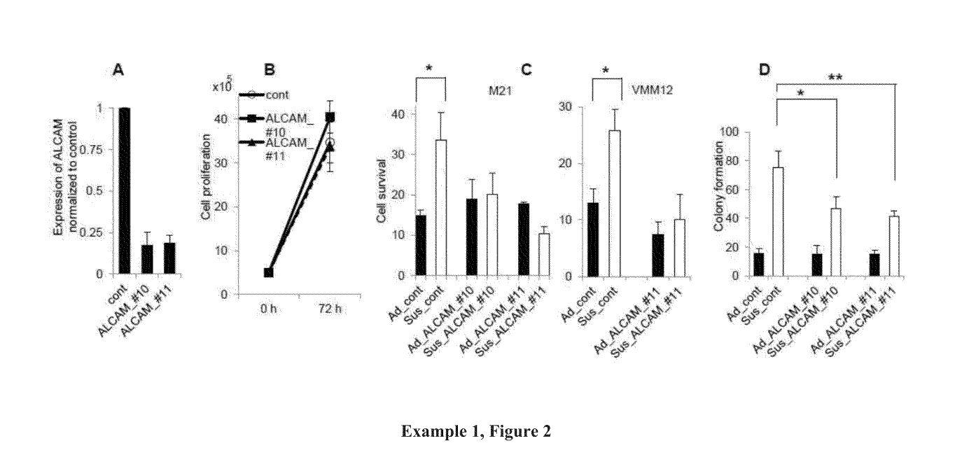

FIG. 2. ALCAM mediates detachment-induced therapy resistance. (A) ALCAM expression by RT-PCR in M21 melanoma cells after transfection with indicated siRNAs. Values are means±SE. (B) Proliferation of M21 cells expressing either ALCAM or control siRNA. 5×10⁵ cells were plated and after 72 h counted. Values are means±SD, (C) Short term survival assays—cells transfected with the indicated siRNAs were treated with chemotherapy (M21:10 μM Ara-C; VMM12:10 μM cisplatin) while adherent (Ad) or suspended (Sus). Cell survival was assayed after 3 days. Values are means±SE, n>3. (D) Adherent or suspended M21 cells were treated with 10 μM Ara-C, then washed and re-plated, and colonies counted after 10 days. Values are means±SD, n=3. **P<0.01, *P<0.05

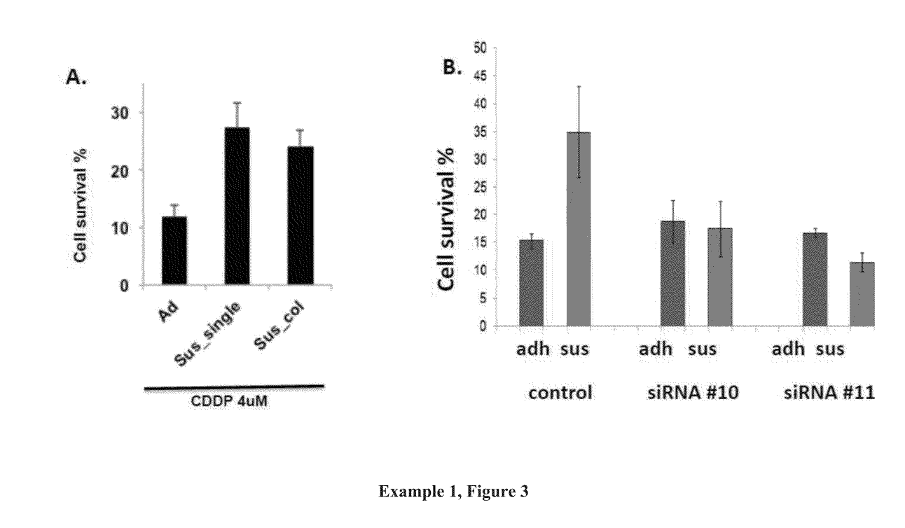

FIG. 3. Single suspended cells still exhibited increased resistance to chemotherapy (see FIG. 3A), indicating that ALCAM does not require cell-cell adhesion to promote survival of suspended cells. (B) M21 and VMM12 cells were subject to chemotherapy for 72 h while adherent or suspended, in the presence or absence of AZN-L50. Survival was then assayed as before. AZN-L50 significantly decreased survival of suspended cells but had no effect on adherent cells (FIG. 3B).

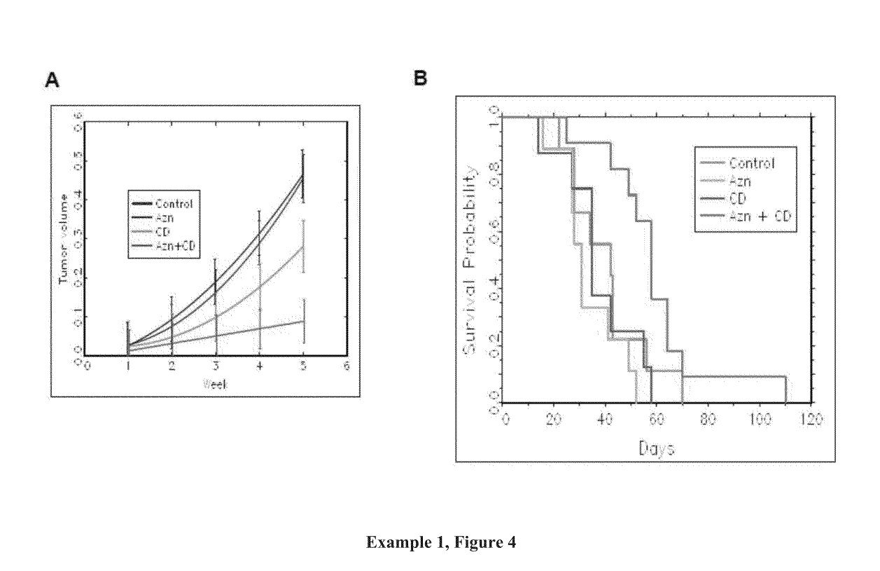

FIG. 4. AZN-L50 enhances chemosensitivity and increases survival in vivo. (A) Mice with palpable subcutaneous tumors were treated with cisplatin alone (CD), AZN-L50 (AZN) alone, or CD plus AZN-L50 (AZN+CD). Mice received 2 treatments 1 week apart. Tumor volume was then followed. Values are means±SE; n=8-11 mice. Statistics: CD vs. CD+AZN, *P<0.0001; AZN vs Control, P=0.96; CD vs Control,*P=0.15; AZN+CD vs Control, P<0.0001; AZN+CD vs AZN,*P=0.08. (B) Survival curves in response to different treatments. Statistics: AZN vs AZN+CD, *P=0.0003; CD vs AZN+CD, *P=0.0067; control vs AZN+CD, *P=0.0257.

### Example 2

FIG. 1, Example 2—Construction and purification of FN-COMP. (A) Schematic representation of FN-COMP. Numbered ovals denote fibronectin type-III repeats (5-11); black rectangle denotes the assembly domain of COMP. C-terminal His6 not shown. (B) Coomassie-stained SDS-PAGE gel. FN-COMP was run under reduced (R) and non-reduced (NR) conditions. (C) Gel filtration chromatography of FN-COMP and fibronectin monomer (FN) as described under Materials and Methods. Standards are: Blue dextran, excluded volume; thyroglobulin, 669,000; catalase, 232,000; ferritin, 440,000; BSA, 67,000 Da.

FIG. 2, Example 2—FN-COMP stimulates signaling in vitro. Either M21 melanoma or mouse 3T3 cells were left adherent (Ad) or detached and kept in suspension (sus) for 1 h. Suspended cells were either treated with of FN-COMP at 40 μg/ml for indicated times or left untreated. Lysates were analyzed by Western blotting using antiphosphotyrosine antibody pY20. Tubulin was used as a loading control. n=2.

FIG. 3, Example 2—FN-COMP sensitizes non-adherent melanoma cells in vitro.

(A) M21, (B) VMM12 or (C) VMM18 melanoma cells were left adherent (Ad) or detached and kept in suspension (Sus) for 1 h. Suspended cells were then treated with of FN-COMP (FNC) at 40 μg/ml for 30-45 min followed by treatment with either 10 Gy X-irradiation (A) or Ara-C at 10 μM (B and C). Cell survival was assayed 72 h later. Values are means } SE, n=3. *P<0.05.

FIG. 4, Example 2—FN-COMP in chemotherapy in vivo. (A) SCID mice were injected with 500 μg of FN-COMP. At the indicated times, plasma was analyzed by western blotting for fibronectin (FN). (B) Mice with palpable subcutaneous tumors were treated with cisplatin (CD), FN-COMP (FNC) alone, or CD plus FNC. Mice received 3 successive treatments 1 week apart. Tumor volumes were then followed. Values are means±SE, n=10-15 mice. For CD vs. CD+FNC: *P<0.0031. The error bars are asymmetric because the analysis was done in the log-scale, and then converted back to the original scale. Qualitatively similar results were obtained in 2 independent experiments.

### DETAILED DESCRIPTION

Abbreviations and Acronyms

a.a.—amino acid

Ad—adherent

ALCAM—activated leukocyte adhesion molecule, also referred to as CD166

AraC—cytosine arabinoside

CD—cisplatin

COMP—short cartilage oligomeric peptide

ECM—extracellular matrix

FN— fibronectin

FNC— fibronectin-COMP

FN-COMP—fibronectin-COMP, also referred to as FNC

GFR—glomerular filtrate rate

i.p.—intraperitoneal

kD—kilodalton

s.c.—subcutaneous

siRNA—small interfering RNA

Sus—suspension

### DEFINITIONS

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As use herein, the terms “administration of” and or “administering” a compound should be understood to mean providing a compound of the invention or a prodrug of a compound of the invention to a subject in need of treatment.

As used herein, the term “aerosol” refers to suspension in the air. In particular, aerosol refers to the particlization or atomization of a formulation of the invention and its suspension in the air.

As used herein, an “agonist” is a composition of matter which, when administered to a mammal such as a human, enhances or extends a biological activity attributable to the level or presence of a target compound or molecule of interest in the mammal.

The term “alterations in peptide structure” as used herein refers to changes including, but not limited to, changes in sequence, and post-translational modification.

An “antagonist” is a composition of matter which when administered to a mammal such as a human, inhibits a biological activity attributable to the level or presence of a compound or molecule of interest in the mammal.

As used herein, “alleviating a disease or disorder symptom,” means reducing the severity of the symptom or the frequency with which such a symptom is experienced by a patient, or both.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

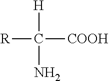

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially an Fab with part of the hinge region (see, FUNDAMENTAL IMMUNOLOGY, 3RD ED., W. E. Paul, ed, Raven Press, N.Y. (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bire, et al., Science 242:423 (1988) and Huston, et al., Proc. Nat'l Acad. Sci. USA 85:5879 (1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin.

Humanized antibodies have been referred to as “reshaped” antibodies. Manipulation of the complementarity-determining regions (CDR) is a way of achieving humanized antibodies. See, for example, Jones, et al., Nature 321:522 (1988) and Riechmann, et al., Nature 332:323 (1988), both of which are incorporated by reference herein. For a review article concerning humanized antibodies, see Winter & Milstein, Nature 349:293 (1991), incorporated by reference herein.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, sputum, mucus, phlegm, tissues, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. One of skill in the art will understand the type of sample needed.

A “biomarker” or “marker” is a specific biochemical in the body which has a particular molecular feature that makes it useful for measuring the progress of disease or the effects of treatment, or for measuring a process of interest.

The term “cancer”, as used herein, is defined as proliferation of cells whose unique trait (loss of normal controls) results in unregulated growth, lack of differentiation, local tissue invasion, and metastasis. Examples include but are not limited to, melanoma, breast cancer, prostate cancer, ovarian cancer, uterine cancer, cervical cancer, skin cancer, pancreatic cancer, colorectal cancer, renal cancer and lung cancer.

As used herein, the term “carrier molecule” refers to any molecule that is chemically conjugated to a molecule of interest.

The term “cell surface protein” means a protein found where at least part of the protein is exposed at the outer aspect of the cell membrane. Examples include growth factor receptors.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

A “computer-readable medium” is an information storage medium that can be accessed by a computer using a commercially available or custom-made interface. Exemplary compute-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

As used herein, a “derivative” of a compound refers to a chemical compound that may be produced from another compound of similar structure in one or more steps, as in replacement of H by an alkyl, acyl, or amino group.

As used herein “detachment-induced chemoresistance” refers to the change in cancer cells that occurs when they become resistant to chemotherapy following a change in their interactions with the extracellular matrix.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, in one embodiment, the term “diagnosis” refers to detecting aberrant ALCAM expression due to cancers expressing ALCAM. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm which is capable of regulating a biochemical pathway.

The term “elixir,” as used herein, refers in general to a clear, sweetened, alcohol-containing, usually hydroalcoholic liquid containing flavoring substances and sometimes active medicinal agents.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

Use of the term “fibronectin peptide” refers to fibronectin or a fragment thereof.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

The term “inhibit a complex,” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means. Compounds or agents of the invention can be administered to a subject by these means when appropriate.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

As used herein, the term “invasive,” or “metastasis” as used herein, refers to any migration of cells, especially to invasive cancer cells or tumor cells. The term applies to normally invasive cells such as wound-healing fibroblasts and also to cells that migrate abnormally. Although the term is not to be limited by any mechanistic rationale, such cells are thought to migrate by defeating the body's means for keeping them sufficiently “in place” to function normally. Such cells are “invasive” if they migrate abnormally within a tissue or tumor, or escape the tissue, or invade other tissues.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

A “receptor” is a compound that specifically binds to a ligand.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

As used herein, the term “malignant” refers to having the properties of anaplasia, penetrance, such as into nearby areas or the vasculature, and metastasis.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “nasal administration” in all its grammatical forms refers to administration of at least one compound of the invention through the nasal mucous membrane to the bloodstream for systemic delivery of at least one compound of the invention. The advantages of nasal administration for delivery are that it does not require injection using a syringe and needle, it avoids necrosis that can accompany intramuscular administration of drugs, and trans-mucosal administration of a drug is highly amenable to self-administration.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides or to peptides shorter than the full length native or mature protein.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “receptor” is a compound that specifically binds to a ligand.

A “ligand” is a compound that specifically binds to a target receptor.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, D.C., p. 574).

A “sample,” as used herein, refers preferably to a biological sample from a subject for which an assay or other use is needed, including, but not limited to, normal tissue samples, diseased tissue samples, sputum, mucus, phlegm, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this invention.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

A “variant”, as described herein, refers to a segment of DNA that differs from the reference DNA. A “marker” or a “polymorphic marker”, as defined herein, is a variant. Alleles that differ from the reference are referred to as “variant” alleles.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### EMBODIMENTS

Previous work showed that many but not all melanomas are inherently sensitive to chemotherapy but become resistant as a consequence of changes in interactions with surrounding extracellular matrix (ECM). These tumors become resistant to chemotherapy when the ECM within the tumor is degraded and integrins are not engaged and signaling.

In the Examples disclosed herein, data are provided from studies investigating the molecular basis for the variability in integrin dependence of chemosensitivity to chemotherapeutic agents in melanoma. DNA microarray analysis of gene expression in melanoma lines showed that activated leukocyte adhesion molecule (ALCAM)/CD166 correlates with detachment-induced chemoresistance. Functional studies showed that expression of this protein was required for chemoresistance in non-adherent cells. Lastly, an antibody to ALCAM reduced chemoresistance in suspended melanoma cells and also improved chemotherapeutic responsiveness in an in vivo model. The present invention therefore encompasses the use of antibodies and other antagonists of ALCAM for treatment of patients with metastatic melanoma and further encompasses the combination of these antagonists with other agents.

The present application discloses a prognostic assay for a protein (ALCAM) whose expression predicts (and in fact causes) responsiveness to integrin stimulation. Patients whose tumors show high expression will therefore benefit from co-administration of a reagent to stimulate integrin signaling together with chemotherapy.

In one embodiment, the present invention comprises obtaining a melanoma sample from a patient and then measuring the ALCAM levels in the sample and determining that the melanoma is chemotherapy resistant when the ALCAM levels are high. In one aspect, the information is used to develop treatment regimens.

The present invention encompasses compositions and methods useful for blocking or inhibiting (ALCAM)/CD166 function, expression, levels and synthesis to overcome its role in chemoresistance of melanomas. In one aspect, (ALCAM)/CD166 is inhibited using an antibody directed against (ALCAM)/CD166. In one aspect, the antibody is a monoclonal antibody. In one aspect, the monoclonal antibody is AZN-L50. In one aspect, ALCAM is inhibited using siRNA directed against ALCAM. In one aspect, the siRNA is siRNA #11 (SEQ ID NO:4) or siRNA #10 (SEQ ID NO:5) One of ordinary skill in the art will appreciate that other types of molecules that can inhibit ALCAM expression, synthesis, levels, and activity are encompassed by the invention, including antisense oligonucleotides and aptamers.

In one embodiment, ALCAM serves as a marker for detachment-induced therapy resistance in melanoma. In one aspect, the resistance is to chemotherapy. In one aspect, the compositions and methods of the invention are useful for improving chemotherapeutic response in melanomas. In one aspect, they are useful for improving the response to other therapies, including, but not limited to, immunotherapy and radiotherapy.

Previous work showed that activating integrin signaling improved chemosensitivity in most melanomas, however, the treatments used in those studies are not suitable for use in human patients. Disclosed herein is a pentameric (i.e., multimeric) fibronectin construct, FN-COMP, was designed for this purpose. It is disclosed herein that FN-COMP activated integrin signaling, increased melanoma chemosensitivity and radiosensitivity in vitro, and significantly improved responses to chemotherapy in vivo. FN-COMP may therefore be useful for treatment of metastatic melanoma in humans.

FN-COMP is a homo-pentameric construct of fibronectin peptides linked by the assembly domain of COMP (amino acid residues 27-84 of COMP) and each fibronectin peptides comprises type III repeats 5-11 of fibronectin.

Fibronectin has the amino acid sequence provided herein (SEQ ID NO:1; NCBI/GenBank accession number P02751, comprising 2386 aa). Use of the term “fibronectin peptide” refers to fibronectin or a fragment thereof. The fibronectin type III repeat region is an approximately 100 amino acid domain, different tandem repeats of which contain binding sites for DNA, heparin and the cell surface. The superfamily of sequences believed to contain FnIII repeats represents 45 different families, the majority of which are involved in cell surface binding in some manner, or are receptor protein tyrosine kinases, or cytokine receptors.

The type III domain regions of fibronectin can be found between residue numbers 610 and 702 (1), 722 and 812 (2), 813 and 904 (3), 909 and 998 (4), 999 and 1098 (5), 1089 and 1175 (6), 1176 and 1266 (7), 1269 and 1361 (8), 1362 and 1449 (9), 1450 and 1543 (10), 1544 and 1635 (11), 1636 and 1723 (12), 1724 and 1817 (13), 1818 and 1904 (14), 1905 and 1995 (15), 2103 and 2197 (16), respectively. The regions “underlined” relate to type III repeats 5-11 of fibronectin, which are used in preparing FN-COMP.

It is disclosed that this reagent stimulates integrin signaling and improves chemosensitivity in vitro and in vivo.

In one aspect, FN-COMP sensitizes melanoma cells to therapy in vivo, improving the outcome. In one aspect, FN-COMP is useful for inhibiting tumor growth.

One of ordinary skill in the art will appreciate that the sequences can be modified with conservative amino acid changes, including, insertions, deletions, and substitutions, and that the valency could be altered as well, as long as the resulting multimer/multimeric complex remains effective. Amino acid changes (fragments and homologs) can be made independently in each fibronectin and in COMP. One of ordinary skill in the art will appreciate that C-terminal groups other than His6 can be used. One of skill in the art will also realize that the pentameric, or other valency, complex can be linked in different ways.

For cancers where integrin-mediated adhesion promotes cell death and growth arrest in response to DNA damage, this treatment will lead to improved therapeutic responses.

In one aspect, the present invention provides administering to a subject in need thereof an antibody such as TS2/16 prior to administration of conventional chemotherapy or radiation, and it can be used in combination with other integrin stimulating agents such as FN-COMP. Schwartz et al. (Clin. Cancer Res., 2008, 14:6193, “Integrin Agonists as Adjuvants in Chemotherapy for Melanoma”) showed that integrin agonists, including TS2/16, can act as adjuvants in chemotherapy for melanoma. In another aspect, the administration of a compound of the invention can be performed at the same time other therapies are being administered. One of ordinary skill in the art will appreciate that numerous techniques are known for determining the best routes, dosages, and timing of administration, as well as how many times administration should occur. The present invention encompasses compounds other than antibodies. In one aspect, an effective compound of the invention is a disintegrin. In one aspect, the compound is contortrostatin. In one aspect, a compound of the invention increases the affinity of an integrin for its ligand(s). The anti-integrin 131 antibody TS2/16 increases the affinity of integrins for matrix proteins and can also crosslink integrins to directly trigger signaling. This antibody therefore increases integrin signaling by two methods. For patients bearing tumors that show decreased sensitivity to therapy when non-adherent, treatment with this antibody will lead to improved therapeutic responses to radiation and chemotherapy.

The present invention provides for a combination therapy using a multimeric complex of the invention and a chemotherapeutic agent or other therapy, such as radiotherapy or immunotherapy. In one aspect, the agent is a drug. In one aspect, the compositions and methods are useful for treating cancer. In one aspect, the compositions and methods are useful for treating metastatic cancer. In another aspect, the compositions and methods are useful for treating any cancer where integrin signaling is involved in the cancer growth. In one aspect, the cancer is melanoma.

Useful agents for treating melanoma include, but are not limited to, Dacarbazine (also called DTIC), Temozolomide, Taxanes, Nab-paclitaxel, Paclitaxel, Nitrosureas, Carmustine (also known as BCNU), Platinum-based agents, Cisplatin, Carboplatin, Vinblastine, Interferon-alpha, Interleukin-2, Ipilimumab, and Vemurafenib.

Platinum-based chemotherapeutic agents useful in the practice of the invention, include, but are not limited to, cisplatin, carboplatin, oxaliplatin, satraplatin, picoplatin, nedaplatin, triplatin, and lipoplatin. In one embodiment, carboplatin is administered intravenously for a time period of at least about 5 minutes. One of ordinary skill in the art will appreciate that the dosage of carboplatin or other platinum-based chemotherapeutic agents is based on health, weight, body size, and response to therapy and that these can be used in conjunction with a known dose calculator. For example, some recent guidelines suggest that the maximum dose is based on a glomerular filtration rate (GFR) estimate that is capped at 125 mL/min for patients with normal renal function and that no higher estimated GFR values should be used.

In general, courses of cisplatin treatment are not given more often than once every four weeks, but can be varied. It can be administered, for example, about once a week, once every two weeks, once every three weeks, once every four weeks, once every five weeks, once every six weeks, once every eight weeks and once every ten weeks. The aforementioned timing regimen is not meant to be exhaustive or exclusive and can include administering the agent at least twice per each time frame, etc.

One of ordinary skill in the art will appreciate that other fragments of fibronectin can be used with the present invention wherein said fragments comprise at least one type III repeat domain and bind with at least one integrin on the target melanoma cells.

One embodiment provides a method to diagnose and distinguish chemoresistant from chemosensitive melanomas, comprising measuring ALCAM in a biological sample from a subject, and diagnosing the sensitivity of the cancer in said subject based on the presence and/or levels of ALCAM in the biological sample. In one embodiment, the subject is mammalian and in another it is human. In another embodiment, the ALCAM is ALCAM protein, miRNA or mRNA. In one embodiment, the detection comprises analyzing the results with an analytical device and program. In one embodiment, the analytical device comprises a computer. In one embodiment, the analytical device comprise a sequence analyzer. In one embodiment, the level of ALCAM protein, miRNA or mRNA is quantified with an analytical device and program. In another embodiment, the ALCAM protein, miRNA, or mRNA is detected using a method selected from the group consisting of ELISA, immunoassay, immunofluorescence, immunohistochemistry, immunoprecipitation, northern blot, western blot, PCR, mass spectrometry and surface Plasmon resonance. In one embodiment, the sample is tissue biopsy.

The present invention further provides compositions and methods useful for precision, personalized medicine. In one embodiment, the present invention provides compositions and methods useful for selecting a subject with cancer who will be responsive to treatment with an antagonist or inhibitor of ALCAM, comprising detecting the presence of ALCAM protein, miRNA or mRNA in a sample from the subject, wherein the presence of ALCAM protein, miRNA or mRNA in the sample indicates that the subject will be responsive to treatment with an antagonist or inhibitor of ALCAM.

Various types of molecules are encompassed within the methods of the invention and are useful for inhibiting the effects of ALCAM. The inhibition may be direct or indirect. For example, useful molecules for inhibiting ALCAM include, but are not limited to, RNAi/siRNA, antisense oligonucleotides, antibodies, aptamers, and other agents and compounds. The compounds of the invention may regulate ALCAM by regulating processes and functions including, but not limited to, gene expression of ALCAM, translation of ALCAM, protein levels of ALCAM, protein degradation of ALCAM, binding of ALCAM with other molecules, as well as both downstream and upstream pathways regulating ALCAM and its functions. By regulating ALCAM is meant regulating ALCAM synthesis, levels, function/activity, binding, and any upstream or downstream pathways regulating ALCAM and its functions.

The present invention also provides compositions and methods useful for treating ALCAM positive cancer.

In one embodiment, the antibody directed against is selected from the group consisting of a single chain antibody, a monoclonal antibody, a bi-specific antibody, a chimeric antibody, a synthetic antibody, a polyclonal antibody, or a humanized antibody, or active fragments or homologs thereof.

In one embodiment, integrin signaling stimulators can be administered to a subject in need thereof. In one embodiment, the integrin signaling stimulator is administered in combination with at least on chemotherapeutic agent. In one embodiment, the chemotherapeutic agent is a platinum-based chemotherapeutic agent.

In one embodiment, the integrin-signaling stimulator is a multimeric peptide construct comprising at least two fibronectin peptides linked with a linker. In one aspect, the multimeric peptide construct comprises at least three, four, five, or six fibronectin peptides. In one aspect, the construct is FN-COMP.

In one aspect, a multimeric peptide construct of the invention can be administered by a route selected from, including, but not limited to, intravenously, intrathecally, locally, intramuscularly, topically, orally, intra-arterially, parenterally, etc.

A multimeric peptide construct can be administered more than once. One of ordinary skill in the art can determine how often to administer the compound, the dose to be used, and what combination of other agents it can be administered with such as chemotherapeutic agents and/or inhibitors of ALCAM levels or expression. One of ordinary skill in the art can also determine if all compounds should be administered simultaneously or not.

In one embodiment, a multimeric peptide construct dosage of about 0.1 mg/kg to about 100 mg/kg can be administered to a subject in need thereof, including whole numbers between 0.1 and 100 and fractions thereof. In one aspect, a multimeric peptide construct dosage of about 1.0 mg/kg to about 75 mg/kg can be administered to a subject. In another aspect, a multimeric peptide construct dosage of about 5.0 mg/kg to about 50 mg/kg can be administered to a subject. In yet another aspect, a multimeric peptide construct dosage of about 10 mg/kg to about 25 mg/kg can be administered to a subject. In a further aspect, a multimeric peptide construct dosage of about 15 mg/kg to about 20 mg/kg can be administered to a subject. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

In one embodiment, a unit dose of multimeric peptide construct can be administered. Other therapeutic agents of the invention can also be administered as unit doses. Kits can be provided with unit doses in a container or syringe or amounts that one of ordinary skill in the art can administer based on a dose per weight, etc.

In one embodiment, a multimeric peptide construct of the invention is administered at least once a day, or once a week, or once month. In one embodiment, a multimeric peptide construct of the invention is administered at least twice a day, or twice a week, or twice a month.

The present invention provides multimeric peptide ligand complexes.

The invention further includes isolated nucleic acids comprising sequences encoding peptides of the invention.

In one embodiment, the useful peptides of the invention are used to prepare multimeric peptide ligand complexes and are modified by adding additional amino acids or substituting amino acids during the synthetic process.

In one aspect, the multimer is selected from the group consisting of a dimer, a trimer, a tetramer, pentamer, hexamer, heptamer, and octamer.

In one aspect, the method provides for the use of an imaging agent selected from the group consisting of a radionuclide, a radiological contrast agent, a paramagnetic ion, a metal, a biological tag, a fluorescent label, a chemiluminescent label, an ultrasound contrast agent and a photoactive agent. One of ordinary skill in the art will understand that the method of detection used will depend on the particular imaging agent used.

In one embodiment, the multimeric peptide ligand complex or an antibody directed against ALCAM comprises an imaging agent selected from the group consisting of a radionuclide, a radiological contrast agent, a paramagnetic ion, a metal, a biological tag, a fluorescent label, a chemiluminescent label, an ultrasound contrast agent and a photoactive agent. In one aspect, the imaging agent is a radionuclide. In one aspect, the radionuclide is selected from the group consisting of ¹¹⁰I, ¹¹¹I, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, and other gamma-, beta-, or positron-emitters. In one aspect, the radionuclide is ¹¹¹In. In one aspect, the imaging agent is detected with a SPECT/CT scanner coupled to a computer, and analyzing imaging data using a program to quantify or compare levels of ALCAM.

The invention further provides kits for diagnosing, detecting, imaging, and treating melanoma.

In one embodiment, the imaging agent is coupled to an antibody directed against ALCAM. In one embodiment, the imaging is coupled to a multimeric fibronectin peptide construct of the invention.

The method comprises administering to a subject a multimeric peptide ligand complex comprising an imaging agent, and detecting the location of melanoma cells using a suitable method for detecting melanomas.

In one aspect, a chemotherapeutic agent is coupled to an antibody directed against ALCAM or is coupled to a multimeric fibronectin peptide construct of the invention, or both.

The method comprises administering to a subject an antibody directed against ALCAM coupled to an imaging agent and detecting the location of melanoma cells comprising ALCAM using a suitable method for detecting and measuring ALCAM.

The invention further provides a method for detecting cancer, diagnosing cancer, monitoring the progression of cancer, or monitoring treatment of a cancer.

Optionally the peptide ligands are modified with conservative amino acid substitutions or additional standard or non-standards are added to enhance distribution or time before degradation, optionally additional amino acids are added as linkers, optionally moieties such as polyethylene are added to the peptide, and each of these are then attached to a chelating agent, optionally via linkers such as flexible amino acid chains, forming a multimeric peptide ligand complex. The chelating agent is useful for attachment of imaging agents. The term “multimeric peptide ligand complex” can refer to a complex with or without an imaging agent, as can the term “multimeric peptide ligand imaging complex” and the terms are meant to be used and interpreted in context. The terms can be qualified by adding the phrase with an imaging agent or the phrase without an imaging agent, or similar phrases.

### Antibodies

Antibodies refer to polypeptides substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

A variety of methods for producing polyclonal and monoclonal antibodies are known in the art. See, e.g., Goding, MONOCLONAL ANTIBODIES; PRINCIPLES AND PRACTICE, Academic Press, 2nd Edition (1986); and Harlow & Lane. A monoclonal antibody directed against or reactive with, for example, human cells expressing a desired antigen is obtained by using combinations of immunogens to immunize mice and screening hybridoma supernatant against cells which express the desired antigen or by a screening assay designed to be specific for monoclonal antibodies directed against the antigen of interest. Useful cell lines for screening for the antibodies of this invention are readily available or obtained. Such cells include the Burkitt's lymphoma cell lines Daudi, and Raji.

Recombinant DNA methodologies can be used to synthesize antibodies of this invention. For example, an antibody portion of an immunotoxin for use in humans is a “humanized” antibody against a cell antigen which contains murine complementarity-determining regions (CDRs) combined with human variable region frameworks and human constant regions.

Humanized (chimeric) antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The humanized chimeric antibody should have the antigen binding specificity of the non-human antibody molecule and the effector function conferred by the human antibody molecule. A large number of methods of generating chimeric antibodies are well known to those of skill in the art (see, e.g., U.S. Pat. Nos. 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431, and 4,975,369). Detailed methods for preparation of chimeric (humanized) antibodies can be found in U.S. Pat. No. 5,482,856.

In another embodiment, this invention provides for fully human antibodies. Human antibodies consist entirely of characteristically human polypeptide sequences. The human antibodies of this invention can be produced in using a wide variety of methods (see, e.g., Larrick et al, U.S. Pat. No. 5,001,065, for review).

The antibody moieties of this invention can be single chain antibodies. In one embodiment, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the invention.

Antibodies directed against proteins, polypeptides, or peptide fragments thereof of the invention may be generated using methods that are well known in the art. For instance, U.S. patent application Ser. No. 07/481,491, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

In one embodiment, antibodies, or antisera, directed against ALCAM or a homolog or fragment thereof, are useful for blocking the activity of ALCAM, including its ability to interact with other molecules or cells.

Fragments of ALCAM may be generated and antibodies prepared against the fragments. Assays are provided herein to determine whether an antibody directed against ALCAM, or a fragment thereof, have the ability to detect ALCAM, to inhibit ALCAM activity, or regulate ALCAM function.

In one embodiment, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the invention.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

In the production of antibodies, screening for the desired antibody can be accomplished by techniques known in the art, e.g., ELISA (enzyme-linked immunosorbent assay). Antibodies generated in accordance with the present invention may include, but are not limited to, polyclonal, monoclonal, chimeric (i.e., “humanized”), and single chain (recombinant) antibodies, Fab fragments, and fragments produced by a Fab expression library.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions that will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method that utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

To ensure that the proteins or peptides obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide can be purified to remove contaminants. In this regard, it will be appreciated that the peptide will be purified to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure peptide obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

### Peptide Modification and Preparation

Peptide preparation is described in the Examples. It will be appreciated, of course, that the proteins or peptides of the invention may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or non-standard synthetic amino acids. The peptides of the invention are not limited to products of any of the specific exemplary processes listed herein.

The invention includes the use of beta-alanine (also referred to as β-alanine, β-Ala, bA, and βA, having the structure:

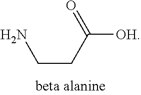

Sequences are provided herein which use the symbol “βA”, but in the Sequence Listing submitted herewith “βA” is provided as “Xaa” and reference in the text of the Sequence Listing indicates that Xaa is beta alanine.

Peptides useful in the present invention, such as standards, or modifications for analysis, may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide may be purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high performance liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

As discussed, modifications or optimizations of peptide ligands of the invention are within the scope of the application. Modified or optimized peptides are included within the definition of peptide binding ligand. Specifically, a peptide sequence identified can be modified to optimize its potency, pharmacokinetic behavior, stability and/or other biological, physical and chemical properties.

### Amino Acid Substitutions

In certain embodiments, the disclosed methods and compositions may involve preparing peptides with one or more substituted amino acid residues.

In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

Pharmaceutical Compositions and Administration

The invention is also directed to methods of administering the compounds of the invention to a subject.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method of treating a subject in need of such treatment is provided. The method comprises administering a pharmaceutical composition comprising at least one compound of the present invention to a subject in need thereof. Compounds identified by the methods of the invention can be administered with known compounds or other medications as well.

The pharmaceutical compositions useful for practicing the invention may be administered to deliver a dose of between 1 ng/kg/day and 100 mg/kg/day.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys. The invention is also contemplated for use in contraception for nuisance animals such as rodents.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In one aspect, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The invention also includes a kit comprising the composition of the invention and an instructional material which describes adventitially administering the composition to a cell or a tissue of a subject. In another embodiment, this kit comprises a (preferably sterile) solvent suitable for dissolving or suspending the composition of the invention prior to administering the compound to the subject.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the invention may, for example, be affixed to a container which contains the multimeric peptide of the invention or be shipped together with a container which contains the peptide. Alternatively, the instructional material may be shipped separately from the container with the intention that the instructional material and the compound be used cooperatively by the recipient.

### Linkers

In one embodiment, linkers are used to link each peptide of the multimer peptide constructs of the invention.

Additionally, modifications encompassed by the invention include introduction of linkers or spacers between the targeting sequence of the binding moiety or binding polypeptide and the detectable label or therapeutic agent. For example, use of such linkers/spacers can improve the relevant properties of the binding peptides (e.g., increase serum stability, etc.). These linkers can include, but are not restricted to, substituted or unsubstituted alkyl chains, polyethylene glycol derivatives, amino acid spacers, sugars, or aliphatic or aromatic spacers common in the art.

For example, suitable linkers include homobifunctional and heterobifunctional cross-linking molecules. The homobifunctional molecules have at least two reactive functional groups, which are the same. The reactive functional groups on a homobifunctional molecule include, for example, aldehyde groups and active ester groups. Homobifunctional molecules having aldehyde groups include, for example, glutaraldehyde and subaraldehyde.

Homobifunctional linker molecules having at least two active ester units include esters of dicarboxylic acids and N-hydroxysuccinimide. Some examples of such N-succinimidyl esters include disuccinimidyl suberate and dithio-bis-(succinimidyl propionate), and their soluble bis-sulfonic acid and bis-sulfonate salts such as their sodium and potassium salts.

Heterobifunctional linker molecules have at least two different reactive groups. Some examples of heterobifunctional reagents containing reactive disulfide bonds include N-succinimidyl 3-(2-pyridyl-dithio)propionate (Carlsson et al., 1978. Biochem. J., 173:723-737), sodium S-4-succinimidyloxycarbonyl-alpha-methylbenzylthiosulfate, and 4-succinimidyloxycarbonyl-alpha-methyl-(2-pyridyldithio)toluene. N-succinimidyl 3-(2-pyridyldithio)propionate is preferred. Some examples of heterobifunctional reagents comprising reactive groups having a double bond that reacts with a thiol group include succinimidyl 4-(N-maleimidomethyl)cyclohexahe-1-carboxylate and succinimidyl m-maleimidobenzoate. Other heterobifunctional molecules include succinimidyl 3-(maleimido)propionate, sulfosuccinimidyl 4-(p-maleimido-phenyl)butyrate, sulfosuccinimidyl 4-(N-maleimidomethyl-cyclohexane)-1-carboxylate, maleimidobenzoyl-5N-hydroxy-succinimide ester.

Furthermore, linkers that are combinations of the molecules and/or moieties described above, can also be employed to confer special advantage to the properties of the peptide. Lipid molecules with linkers may be attached to allow formulation of ultrasound bubbles, liposomes or other aggregation based constructs. Such constructs could be employed as agents for targeting and delivery of a diagnostic reporter, a therapeutic agent (e.g., a chemical “warhead” for therapy), or a combination of these.

Constructs employing dimers, multimers, or polymers of one or more peptide ligands of the invention are also contemplated. Indeed, there is ample literature evidence that the binding of low potency peptides or small molecules can be substantially increased by the formation of dimers and multimers. Thus, dimeric and multimeric constructs (both homogeneous and heterogeneous) are within the scope of the instant invention. The polypeptide sequences in the dimeric constructs can be attached at their N- or C-terminus or the N-epsilon nitrogen of a suitably placed lysine moiety (or another function bearing a selectively derivatizable group such as a pendant oxyamino or other nucleophilic group), or can be joined together via one or more linkers (e.g., those discussed herein) employing the appropriate attachment chemistry. This coupling chemistry can include amide, urea, thiourea, oxime, or aminoacetylamide (from chloro- or bromoacetamide derivatives, but is not so limited). For example, methods to prepare dimeric or multimeric constructs of Plec1 binding polypeptides of the invention include at least those discussed below.

Linkers can also be used for attachment to a chelating agent.

### Therapeutic Agents

In other embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes or other agents may be used as adjunct therapies when using the multimeric peptide ligand complexes described herein. Drugs useful in the invention may, for example, possess a pharmaceutical property selected from the group consisting of antimitotic, antikinase, alkylating, antimetabolite, antibiotic, alkaloid, anti-angiogenic, pro-apoptotic agents, and combinations thereof.

Techniques for detecting and measuring these agents are provided in the art or described herein.

Aptamers

The present invention is also directed to useful aptamers. In one embodiment, an aptamer is a compound that is selected in vitro to bind preferentially to another compound (in this case the identified proteins). In one aspect, aptamers are nucleic acids or peptides, because random sequences can be readily generated from nucleotides or amino acids (both naturally occurring or synthetically made) in large numbers but of course they need not be limited to these. In another aspect, the nucleic acid aptamers are short strands of DNA that bind protein targets. In one aspect, the aptamers are oligonucleotide aptamers. Oligonucleotide aptamers are oligonucleotides which can bind to a specific protein sequence of interest. A general method of identifying aptamers is to start with partially degenerate oligonucleotides, and then simultaneously screen the many thousands of oligonucleotides for the ability to bind to a desired protein. The bound oligonucleotide can be eluted from the protein and sequenced to identify the specific recognition sequence. Transfer of large amounts of a chemically stabilized aptamer into cells can result in specific binding to a polypeptide of interest, thereby blocking its function. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Aptamers offer advantages over other oligonucleotide-based approaches that artificially interfere with target gene function due to their ability to bind protein products of these genes with high affinity and specificity. However, RNA aptamers can be limited in their ability to target intracellular proteins since even nuclease-resistant aptamers do not efficiently enter the intracellular compartments. Moreover, attempts at expressing RNA aptamers within mammalian cells through vector-based approaches have been hampered by the presence of additional flanking sequences in expressed RNA aptamers, which may alter their functional conformation.

The idea of using single-stranded nucleic acids (DNA and RNA aptamers) to target protein molecules is based on the ability of short sequences (20 mers to 80 mers) to fold into unique 3D conformations that enable them to bind targeted proteins with high affinity and specificity. RNA aptamers have been expressed successfully inside eukaryotic cells, such as yeast and multicellular organisms, and have been shown to have inhibitory effects on their targeted proteins in the cellular environment.

In binding assays, the interaction is binding and the complex formed can be isolated or detected in the reaction mixture. In a particular embodiment, one of the peptides of the complexes described herein, or the test compound or drug candidate is immobilized on a solid phase, e.g., on a microtiter plate, by covalent or non-covalent attachments. Non-covalent attachment generally is accomplished by coating the solid surface with a solution of the peptide and drying. Alternatively, an immobilized antibody, e.g., a monoclonal antibody, specific for the peptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

The present invention also encompasses pharmaceutical and therapeutic compositions comprising the multimeric peptide ligand complexes of the present invention. More particularly, such compounds can be formulated as pharmaceutical compositions using standard pharmaceutically acceptable carriers, fillers, solublizing agents and stabilizers known to those skilled in the art.

The present invention further provides a pharmaceutical preparation comprising one or more of the multimeric peptide ligands or complexes of the invention. The concentration of the polypeptide in the pharmaceutical composition can vary widely, i.e., from less than about 0.1% by weight, usually being at least about 1% by weight to as much as 20% by weight or more.

The composition may comprise a pharmaceutically acceptable carrier in addition to the active ingredient. The pharmaceutical carrier can be any compatible, non-toxic substance suitable to deliver the peptides o to the patient. For polypeptides, sterile water, alcohol, fats, waxes, and inert solids may be used as the carrier. Pharmaceutically acceptable adjuvants, buffering agents, dispersing agents, and the like, may also be incorporated into the pharmaceutical compositions.

Methods of producing pharmaceutical compositions comprising polypeptides are described in U.S. Pat. Nos. 5,789,543 and 6,207,718. The preferred form depends on the intended mode of administration and therapeutic application.

In one embodiment, the present compositions comprising multimeric peptides are administered by injection. The parenteral route for administration of the polypeptide is in accordance with known methods, e.g. injection or infusion by intravenous, intraperitoneal, intramuscular, intra-arterial, subcutaneous, or intralesional routes. The protein or polypeptide may be administered continuously by infusion or by bolus injection. A typical composition for intravenous infusion could be made up to contain 10 to 50 ml of sterile 0.9% NaCl or 5% glucose optionally supplemented with a 20% albumin solution and between 10 ug and 50 mg, preferably between 50 ug and 10 mg, of the polypeptide. A typical pharmaceutical composition for intramuscular injection would be made up to contain, for example, 1-10 ml of sterile buffered water and between 10 ug and 50 mg, preferably between 50 ug and 10 mg, of the polypeptide of the present invention. Methods for preparing parenterally administrable compositions are well known in the art and described in more detail in various sources, including, for example, Remington's Pharmaceutical Science (15th ed., Mack Publishing, Easton, Pa., 1980) (incorporated by reference in its entirety for all purposes).

Other techniques known in the art may be used in the practice of the present invention.

The invention is now described with reference to the following Examples. Without further description, it is believed that one of ordinary skill in the art can, using the preceding description and the following illustrative examples, make and utilize the present invention and practice the claimed methods. The following working examples therefore, are provided for the purpose of illustration only and specifically point out the preferred embodiments of the present invention, and are not to be construed as limiting in any way the remainder of the disclosure. Therefore, the examples should be construed to encompass any and all variations which become evident as a result of the teaching provided herein.

### Examples

### Example 1

### Results

Identification of ALCAM as Marker for Detachment-Induced Therapy Resistance in Melanoma

We previously established that chemotherapy is less effective on suspended than on adherent cells in the majority of melanoma isolates [Schwartz, 2008]. This was the case for M21, VMM12, VMM5A, VMM15, and VMM1 lines, whereas in VMM18 cells, chemosensitivity was insensitive to integrin-mediated adhesion [Schwartz, 2008]. Furthermore, among the cell lines where therapeutic responses were adhesion-dependent, the magnitude of the difference varied substantially. In order to identify genes whose expression correlates with integrin-dependent responsiveness to chemotherapy, we performed microarray analyses on these 6 cell lines and screened for genes whose expression varied with the degree of integrin-dependence. The results were then filtered for genes whose function was linked to cell adhesion or cell survival/apoptosis. Among the identified genes, activated leukocyte cell adhesion molecule/CD166, hereafter termed ALCAM, was the top candidate, being highly expressed in the lines where loss of mediated adhesion induced chemoresistance but was nearly absent in VMM18 cells where chemosensitivity was adhesion-independent (FIG. 1A and data not shown). Real-time PCR confirmed that ALCAM mRNA was expressed a much higher levels in M21, VMM12, VMM5A, VMM15, and VMM1 melanomas compared to VMM18 (FIG. 1B).

We next tested this correlation in four additional melanoma lines. Analysis of ALCAM expression by real time PCR showed a range of expression across these cell lines (FIG. 1C). Survival after chemotherapy treatment of adherent and suspended cells was assayed as before. We defined detachment-induced resistance (“delta”) as the fold difference in survival between suspended versus adherent cells. Note that chemosensitivity per se does not correlate with delta (Schwartz 2008 and unpublished data). For these cells, delta again correlated with ALCAM expression (FIG. 1D). These data suggest that ALCAM may be a useful marker to predict whether chemosensitivity in a given tumor is dependent on adhesion to the extracellular matrix.

### ALCAM is Required for Detachment-Induced Chemoresistance

We next asked whether ALCAM might play a causal role in chemoresistance induced by loss of integrin-mediated cell adhesion. We therefore designed and tested several siRNA sequences. When M21 and VMM12 cells were transfected with these siRNAs, two of the sequences greatly reduced expression of ALCAM, whereas the control, scrambled sequence had no effect (FIG. 2A). Depletion of ALCAM did not affect growth in culture (FIG. 2B), indicating that it is not required for viability or cell cycle progression. We next examined chemosensitivity in adherent and suspended M21 and VMM12 melanoma lines. Cells transfected with two distinct ALCAM siRNAs (#10 or #11), or control siRNA, were treated with chemotherapy for 3 days, then survival assayed by re-plating on fibronectin. Both siRNAs drastically reduced cell survival in suspended cells but had no effect in adherent cells (FIG. 2C). Control siRNA had no effect in either condition. Clonogenic survival assays confirmed that depletion of ALCAM significantly reduced survival in cells treated with chemotherapy in suspension but had no effect on those treated while adherent (FIG. 2D). These data indicate that ALCAM is required for detachment-induced chemoresistance, which strongly reinforces the correlation between its expression and integrin-dependence of chemosensitivity in melanomas.

### Effects of ALCAM Antibody

ALCAM can mediate homophilic adhesion, as well as heterophilic adhesion to CD6, both of which are blocked by a monoclonal antibody, AZN-L50 (van Kempen J Biol Chem. 2001; 276:25783-90; Zimmerman Blood. 2006; 107:3212-20). However, single suspended cells still exhibited increased resistance to chemotherapy (FIG. 3A), indicating that ALCAM does not require cell-cell adhesion to promote survival of suspended cells. However, there is evidence that AZN-L50 alters ALCAM clustering in the plasma membrane (van Kempen 2001), thus, we considered whether it might also inhibit ALCAM-dependent detachment-induced chemoresistance. M21 and VMM12 cells were subject to chemotherapy for 72 h while adherent or suspended, in the presence or absence of AZN-L50. Survival was then assayed as before. AZN-L50 significantly decreased survival of suspended cells but had no effect on adherent cells (FIG. 3B).

We next addressed whether AZN-L50 enhances therapeutic responses in vivo. Mice bearing subcutaneous M21 tumors were treated with cisplatin alone, AZN-L50 alone, or cisplatin plus AZN-L50, receiving two treatments ˜1 week apart. Tumor growth was then monitored (see FIG. 4). Neither AZN-L50 alone nor cisplatin alone had significant effects on tumor growth (AZN-L50 vs. control: P=0.96; cisplatin vs. control: P=0.15). The combined treatment, however, dramatically decreased tumor size. When compared with sum of the individual effects of cisplatin and AZN-L50, the combined treatment was clearly synergistic (cisplatin vs+AZN-L50+cisplatin: P=0.0001). In addition to the decrease in tumor size, tumor free survival also increased following AZN-L50 plus cisplatin treatment (FIG. 4; cisplatin vs+AZN-L50+cisplatin: *P=0.0067).

### Discussion

We first utilized microarrays to identify genes that could predict detachment-induced therapy resistance in melanomas. ALCAM message level showed the highest association with detachment-induced therapy-resistance. Moreover, depletion of ALCAM specifically improved therapy responsiveness in suspended cells while having no effect in adherent cells. ALCAM is therefore functionally as well as statistically linked to the requirement for integrin-mediated adhesion in cell responses to therapy. This result is of particular interest in that ALCAM expression also correlates with invasiveness and poor prognosis of melanoma [Weidle, 2010; van Kempen, 2000]. The data therefore raise the intriguing idea that suspension-induced chemoresistance is a feature of more aggressive tumors. Therapies that are effective in these advanced tumors would be especially useful in the clinic.

ALCAM is an immunoglobulin superfamily transmembrane protein expressed on hematopoietic cells, neuronal cells, mesenchymal stem cells, and bone marrow stromal cells. Elevated expression of ALCAM in melanoma, prostate carcinoma, breast cancer, colorectal carcinoma, bladder cancer, and esophageal squamous cell carcinoma correlates with invasiveness and poor prognosis (reviewed in Weidle, 2010). Analysis of ALCAM in a series of 121 biopsies of melanocytic lesions of all stages, from common nevus to metastases, showed a very strong correlation between ALCAM positivity and tumor progression [van Kempen, 2000]. Additionally, random phage display screens identified ALCAM as a readily accessible therapeutic target on multiple cancer cells [Piazza, 2005; Liu, 2007]. ALCAM has recently been recognized as a novel biomarker associated with chemoresistance in pancreatic, colon, fibrosarcoma and breast cancer [Kahlert, 2009; Piazza, 2005]. For example, ALCAM silencing using RNAi had no effects on growth or invasion of pancreatic cancer cells but reduced cell adhesion and induced chemoresistance (Hong et al., 2010). In neuroendocrine tumor cell lines, silencing of ALCAM decreased cell growth.

These observations are consistent with our observation that ALCAM both predicts and mediates chemoresistance, and raises the possibility that detachment-induced chemoresistance plays a role in other therapy-resistant tumors.

It is of particular interest that, as well as being linked to cancer aggressiveness, ALCAM has been identified as a stem cell marker in several systems (Jiao PLoS One. 2012; 7(8):e42564; Kijima Neuro Oncol. 2012; 14:1254-64; Levin, Gastroenterology. 2010; 139:2072-2082). These correlations suggest that detachment-induced chemoresistance may correlate with “stemness”. This finding is all the more intriguing in light of recent evidence that a large fraction of melanoma cells express stem cell markers and have the potential to be tumor initiating cells [Kuphal, 2009; Quintana, 2008]. Cancer initiating or progenitor cells are typically grown as non-adherent “spheres”, which promotes maintenance of “stemness” (Su Biomaterials. 2013; 34:3215-22; Dontu 2003 Genes Dev 17:1253-70). Moreover, both cancer initiating cells and normal stem cells are highly chemoresistant (Abdullah Clin Transl Med. 2013, 2:3; Pollak Curr Hematol Rep. 2003; 2:341-7). The current data therefore suggest possible connections between microenvironment, stemness, and chemoresistance. Further work will be required to test these intriguing connections and elucidate mechanisms. Whatever its mechanistic basis, the identification of a method to specifically target this difficult-to-treat subpopulation of cells may be clinically useful.

### Experimental Procedures

### Example 1

### Materials

cis-Dichlorodiamine platinum(II) (cisplatin) and 1-β-D-arabinofuranosylcytosine (Ara-C) were obtained from Sigma. The TS2/16 hybridoma was obtained from ATCC and purified IgG prepared in the University of Virginia hybridoma facility (Schwartz et al., 2008, Clin. Cancer Res., 14:6193). The antibody is specific for the human 131 integrin (Arroyo et al., 1992, J. Cell Biol., 117:659). Contortrostatin—Unlike other disintegrins, contortrostatin is dimeric and efficiently crosslinks integrins to promote signaling. Contortrostatin binds to both αv integrins and α5β1, which are both generally expressed on melanoma cells. Tyrosine phosphorylation of integrin targets such as focal adhesion kinase and p130cas are strongly increased and are observed at nM concentrations.

### Cell Culture.

The human melanoma cell lines; M21, VMM12, VMM15, VMM1, VMM5A, VMM18 were cultured as described [Molhoek, 2008; Schwartz, 2008]. RPMI7951, A208, and A375.S2 were cultured in DMEM/F12 medium supplemented with 5% heat inactivated fetal bovine serum, 15 mM HEPES and penicillin-streptomycin. FM3 cells were grown in DMEM supplemented with 10% fetal bovine serum and penicillin-streptomycin. Cell survival assays and colony assay were performed as described before [Schwartz, 2008].

### Quantitative RT-PCR

To quantify mRNA levels, total RNA was isolated using the Qiagen RNeasy Kit (Valencia, Calif.). cDNAs were made using the iScript cDNA Synthesis kit (BioRad). Real time RT-PCR was performed using the BioRad iCycler and Sybr Green Master Mix kit. Primers were: ALCAM forward primer: 5-TCTTAGCACCTGGCGTTTCA-3 (SEQ ID NO:2); ALCAM reverse primer: 5-CGACCCTCTGTTTCCAGGAG-3 (SEQ ID NO:3) and human b-actin [Murphree, 2005]. Data were analyzed as described, using the relative expression method [Livak, 2001].

### Small Interfering RNA Experiments.

Melanoma cells were transfected with Small interfering RNA (SiRNA) using the Lipofectamine™ RNAiMAX transfection reagent (Invitrogen) according to the manufacturer's protocol and double knock downs were done in two consecutive days to ensure high knock-down efficiency. ALCAM siRNA #11 and control siRNA sequences were described before [Hong, 2010; 5′-CACCTGCTCGGTGACATATTA-3′ (SEQ ID NO:4); the control siRNA sequence was: UUCUCCGAACGUGUCACGU (SEQ ID NO:6)]. siRNA oligonucleotides that target ALCAM (ALCAM siRNA #10: 5-GGAAACUAUGUCUGCGAAA-3; SEQ ID NO:5), ALCAM siRNA #11 (SEQ ID NO:4) and control siRNA (SEQ ID NO:6) were purchased from Invitrogen and re-suspended according to the manufacturer's instructions.

### Gene Expression Analysis.

Microarray analysis was conducted in collaboration of DNA Sciences Core in the University of Virginia. Affymetrix Genechips, Human Genome X3P, were purchased and processed essentially as previously described [Gallagher, 2003]. Briefly, total RNAs from various cell lines were extracted using Qiagen RNeasy kits. Biotin-labeled cRNA was prepared from approximately 2 ug of total RNA and hybridized to the oligonucleotide array chips. After washing in a fluidic station, the arrays were scanned with a 2.5 micron resolution Affymetrix Microarray Scanner (Affymetrix, Santa Clara, Calif.). Data quality assessment and analysis were performed using combination of the Affymetrix Microarray Analysis Suite 5.0 (MAS 5.0, Affymetrix, Santa Clara, Calif.) and the dChip software [Li, 2001; Schadt, 2001; and R/BioConductor (Ihaka, 1996)]. For data mining, GenMAPP database and Ingenuity (Ingenuity Systems, Inc, Redwood City, Calif.) were used in conjunction with Together to reveal a global gene expression profile by integrating the annotation of the gene ontology and pathway studies.

### Mouse Tumor Growth.

Tumor generation and measurement was done as described [Schwartz, 2008]. Briefly male SCID mice (Charles River) were injected s.c. with 100,000 M21 cells. When tumors were first palpable, mice were injected I.P with cisplatin (8 mg/kg: first week and 3.5 mg/kg: second week), AZN-L50 (100 μg/mouse) or both for 2 consecutive weeks. The tumor volumes were recorded weekly. All animal procedures were done according to our institutional Animal Care and Use Committee guidelines.

Statistical Analysis.

All statistical analyses were done as described [Schwartz, 2008]. Briefly repeated measures models were used to compare tumor growth in control, cisplatin alone, AZN-L50 alone and cisplatin+AZN-L50 groups. These models account for the correlations among measurements taken from the same animal over time. We used linear models for the log-transformed tumor growth and the assumption that the correlation between measurements decreases with the amount of time between the measurements. F-tests based on contrasts were used to make specific comparisons between groups. All analyses were carried out in SAS 9.2 and GAUSS 10.0.

### Example 2

### Multimeric Compounds and Methods to Improve Chemosensitivity in Cancer Therapy

### Experimental Procedures

### Example 2

### Materials

cis-Dichlorodiamine platinum(II) (cisplatin) and 1-β-D-arabinofuranosylcytosine (Ara-C) were obtained from Sigma. Monoclonal anti-β-tubulin antibody (E7) was obtained from the Developmental Studies Hybridoma Bank (Iowa City, Iowa). Human fibronectin hybridoma was generously provided by Dr. Ken Yamada, NIH and produced at University of Virginia hybridoma facility.

Cell Culture and Irradiation.

The human melanoma cell lines; M21, VMM12, VMM18 were cultured as described [7, 19]. Cell survival assays and colony assay were performed as described [7]. Cells were irradiated using an X-ray irradiator at a dose rate of 3.3 Gy/min.

Construction of FN-COMP.

To generate FN-COMP-6xHis, DNA fragments encoding the fibronectin type-III repeats 5-11 and the assembly domain of COMP (amino acids 27-84) [14] were produced by PCR using full-length, human cDNA sequences as a template. Fibronectin as used herein has the amino acid sequence of SEQ ID NO:1 (NCBI/GenBank accession number P02751), comprising 2386 aa.

The type III domain regions of fibronectin can be found between residue numbers 610 and 702 (1), 722 and 812 (2), 813 and 904 (3), 909 and 998 (4), 999 and 1098 (5), 1089 and 1175 (6), 1176 and 1266 (7), 1269 and 1361 (8), 1362 and 1449 (9), 1450 and 1543 (10), 1544 and 1635 (11), 1636 and 1723 (12), 1724 and 1817 (13), 1818 and 1904 (14), 1905 and 1995 (15), 2103 and 2197 (16), respectively.

The PCR products were flanked by NcoI and SalI (for FN) or SalI and BglII sites (for COMP) and were both ligated in-frame with the 6x-His tag of pET-28c linearized with NcoI and BamHI.

Western Blot Analysis.

Cells were lysed as previously described [20]. Proteins were separated on SDS-PAGE gels, transferred, blocked, and probed with the indicated primary antibodies. Blots were washed, incubated with secondary antibodies and developed using ECL reagents (Pierce) and film (Kodak). FN-COMP under reducing and non-reducing condition was loaded on SDSPAGE gels and was subjected electrophoresis. Gels were stained with Coomassie Blue to visualize the bands.

Gel Filtration.

Gel filtration was performed on a Pharmacia Biotech/GE Healthsciences Superdex 200 column (10 mm×290 mm) operated on an Amersham/GE Healthcare AKTA FPLC at room temperature. The buffer was 30 mM Tris pH 9, 250 mM NaCl, 10% glycerol, 5 mM MgCl2, flow rate 0.25 ml/min and detection was absorbance at 280 nm.

Mouse Tumor Growth, Measurement of Primary Tumor Growth and Collection of Blood.

Tumor generation in 6-8 wk old male SCID mice (Charles River Laboratories) and tumor measurement were done as described [7]. When tumors were first palpable, mice were injected intraperitoneally (i.p.) with cisplatin (8 mg/kg: first week and 3.5 mg/kg: second and third weeks), FN-COMP (500 μg/mouse) or both for 3 consecutive weeks. The tumor volumes were recorded weekly. To assay levels in the blood, SCID mice with no tumors were injected intraperitoneally with FN-COMP (500 μg/mouse). Blood was collected at 0, 3, 6, or 24 h post injection. Plasma was subjected to Western blotting with the indicated antibodies. All animal procedures were done according to our institutional Animal Care and Use Committee guidelines.

Statistical Analysis.

Statistical analyses were done as described [7]. Briefly, repeated measures models were used to compare tumor growth in control, cisplatin alone, FN-COMP alone and cisplatin+FN-COMP groups. These models account for the correlations among measurements taken from the same animal over time. We used linear models for the log-transformed tumor growth and the assumption that the correlation between measurements decreases with the amount of time between the measurements. F-tests based on contrasts were used to make specific comparisons between groups. All analyses were carried out in SAS 9.2 and GAUSS 10.0.

### Results

### Example 2

### Protein Expression, Purification, and Characterization

Numerous studies have shown that clustering is the key event in integrin signaling [10]. Multivalent integrin-binding reagents can cluster integrins to mimic adhesion to extracellular matrices [11]. Multivalency also increases affinity for cells so that reagents are effective at low concentrations [12-14]. We therefore constructed a pentavalent fibronectin (FN-COMP; FIG. 1A) in which the integrin binding region (type III repeats 5-11) was linked to the short cartilage oligomeric peptide (COMP) that mediates pentamer formation; this protein contains the region of fibronectin that binds to integrins α5β1, αVβ3 and α4β1, all of which are commonly expressed on melanomas. A distinct fibronectin pentamer was previously found to bind and localize with α5β1 integrin. The fusion was expressed in bacteria and purified via a C-terminal His6 tag. SDS-PAGE of the purified proteins under reducing and non-reducing conditions gave rise to major bands of the expected molecular mass (FIG. 3B) [14, 15]. The fusion was expressed in bacteria and purified via a C-terminal His6 tag. SDS-PAGE of the purified proteins under reducing and non-reducing conditions gave rise to major bands of the expected molecular mass (FIG. 1B). Gel filtration chromatography confirmed that the protein formed a large complex that ran as a single species, demonstrating efficient oligomerization (FIG. 1C). Both the monomer and the pentamer migrate at positions that are larger than their expected molecular masses, which could be due to an elongated conformation or to additional protein-protein interactions, as previously suggested [14].

Characterization of Fibronectin Pentamer

FN-COMP contains the region of fibronectin that binds to integrins α5β1 and αVβ3, both of which are commonly expressed on melanomas [16]. A distinct fibronectin pentamer was previously found to bind and localize with α5β1 integrin [14]. Next we tested the ability of this construct to stimulate integrin signaling. Addition of this protein to suspended cells triggered increased tyrosine phosphorylation of bands around 60, 75 and 125 kD (FIG. 2), which are characteristic of integrin signaling [17].

Fibronectin Pentamer Sensitizes Melanoma Cells to Therapy In Vitro

We next tested whether stimulating integrin signaling with FN-COMP would enhance the response to therapy in non-adherent human melanoma cells. These experiments used M21 and VMM12 cells, two melanoma lines that show decreased chemosensitivity after detachment [7]. We found that addition of FN-COMP to non-adherent melanoma cells improved chemo and radiosensitivity (FIGS. 3A and B). By contrast, FN-COMP showed no effect on VMM18 cells, a line where chemosensitivity is independent of cell adhesion (FIG. 3C). FN-COMP is therefore able to enhance therapy sensitivity of melanoma cells that show detachment-induced chemoresistance. Since relatively modest differences in chemosensitivity in vitro can have major effect on patient survival [18], these data support the idea that restoring therapy sensitivity in non-adherent melanomas may be clinically relevant.

### Fibronectin Pentamer Decreases Tumor Growth In Vivo

Having validated FN-COMP in vitro, we next tested whether this reagent would enhance therapeutic responses in vivo. We first assessed the ability of FN-COMP to enter the blood and its stability after intraperitoneal injection into mice. Initial studies showed that it appeared in the blood by 3 h and was stable for at least 24 h after injection (FIG. 4A). Next, mice bearing subcutaneous M21 tumors were treated with cisplatin alone, FN-COMP alone, or cisplatin plus FN-COMP, receiving three treatments ˜1 week apart. Tumor growth was then monitored (FIG. 4B). Neither FN-COMP alone nor cisplatin alone had significant effects on tumor growth (FN-COMP vs. control: P=0.177; cisplatin vs. control: P=0.432). The combined treatment, however, dramatically decreased tumor size. When compared with sum of the individual effects of cisplatin and FN-COMP, the combined treatment was clearly synergistic (cisplatin vs FN-COMP+cisplatin: P=0.0031).

### Discussion

### Example 2

In this study, we developed a new reagent to address therapy resistance in melanoma. A previous report showed that in mouse xenografts of human melanoma, combining chemotherapy with reagents that stimulate integrin signaling (e.g. contortrostatin, TS2/16) improved therapeutic responses [7]. Although the reagents used in that study are not suitable for use in patients, the results were important because they showed that loss of integrin-mediated adhesion is rate limiting for therapeutic response. We describe here a pentameric version of the cell-binding region of fibronectin that stimulates integrin signaling to enhance chemosensitivity. Like soluble fibronectin circulating in the blood, this construct should only bind activated integrins, thus, should interact minimally with circulating platelets and leukocytes. However, in cells containing activated, unoccupied integrins, FN-COMP should bind and crosslink the receptors to stimulate signaling. FN-COMP was readily purified in large quantity and, when added to suspended melanoma cells, stimulated integrin signaling and enhanced chemosensitivity in vitro. To test its efficacy in vivo, mice with subcutaneous tumors were treated with cisplatin and/or FN-COMP. While FN-COMP alone or cisplatin alone had little effect, treating cisplatin together with FN-COMP caused substantial and highly significant decreases in tumor size.

Remarkably, dramatic results were observed after only 3 treatments, compared with regimens of many months for chemotherapy in patients. Not unexpectedly, tumors resumed growth ˜5 weeks after treatment ended. However, the substantial effects on an otherwise therapy-resistant line with this limited treatment regimen are impressive and suggest that stronger responses may be obtained using longer protocols. Side effects in the mice were not observed. These data therefore strongly argue that loss of integrin-mediated adhesion is an important causative factor in therapy resistance of melanoma and that FN-COMP can reverse this effect.

In summary, combining chemotherapy or radiotherapy with FN-COMP to stimulate integrin signaling, offers a new direction for improving therapeutic outcomes in the treatment of otherwise intractable metastatic melanomas. Much further work to test FN-COMP efficacy and specificity in clinical cancer therapy will be required.

### Example 2

### Bibliography

1. Zalaudek, I., et al., Diagnosis and treatment of cutaneous melanoma: a practical guide. Skinmed, 2003. 2(1): p. 20-31; quiz 32-3.
2. Kasper, B., et al., Novel treatment strategies for malignant melanoma: a new beginning? Crit Rev Oncol Hematol, 2007. 62(1): p. 16-22.
3. Schwartz, M. A. and R. K. Assoian, Integrins and cell proliferation: regulation of cyclin dependent kinases via cytoplasmic signaling pathways. J Cell Sci, 2001. 114 (Pt 14): p. 2553-60.
4. Frisch, S. M. and R. A. Screaton, Anoikis mechanisms. Curr Opin Cell Biol, 2001. 13(5): p. 555-62.
5. Truong, T., et al., Modulation of DNA damage-induced apoptosis by cell adhesion is independently mediated by p53 and c-Abl. Proc Natl Acad Sci USA, 2003. 100(18): p. 10281-6.
6. Lewis, J. M., T. N. Truong, and M. A. Schwartz, Integrins regulate the apoptotic response to DNA damage through modulation of p53. Proc Natl Acad Sci USA, 2002. 99(6): p. 3627-32.
7. Schwartz, M. A., et al., Integrin agonists as adjuvants in chemotherapy for melanoma. Clin Cancer Res, 2008. 14(19): p. 6193-7.
8. Van Duinen, C. M., G. J. Fleuren, and J. A. Bruijn, The extracellular matrix in pigmented skin lesions: an immunohistochemical study. Histopathology, 1994. 24(1): p. 33-40.
9. Lugassy, C., et al., Ultrastructural and immunohistochemical studies of the periendothelial matrix in human melanoma: evidence for an amorphous matrix containing laminin. J Cutan Pathol, 1999. 26(2): p. 78-83.
10. Schwartz, M. A., M. D. Schaller, and M. H. Ginsberg, Integrins: emerging paradigms of signal transduction. Annu Rev Cell Dev Biol, 1995. 11: p. 549-99.
11. Gresham, H. D., S. P. Adams, and E. J. Brown, Ligand binding specificity of the leukocyte response integrin expressed by human neutrophils. J. Biol. Chem., 1992. 267(20): p. 13895-902.
12. Garanger, E., et al., Multivalent RGD synthetic peptides as potent alphaVbeta3 integrin ligands. Org. Biomol. Chem., 2006. 4(10): p. 1958-65.
13. Thumshirn, G., et al., Multimeric cyclic RGD peptides as potential tools for tumor targeting: solid-phase peptide synthesis and chemoselective oxime ligation. Chemistry, 2003. 9(12): p. 2717-25.
14. Coussen, F., et al., Trimers of the fibronectin cell adhesion domain localize to actin filament bundles and undergo rearward translocation. J. Cell Sci., 2002. 115 (Pt 12): p. 2581-90.
15. Tomschy, A., et al., Homophilic adhesion of E-cadherin occurs by a co-operative two step interaction of N-terminal domains. EMBO J, 1996. 15(14): p. 3507-14.
16. Gehlsen, K. R., G. E. Davis, and P. Sriramarao, Integrin expression in human melanoma cells with differing invasive and metastatic properties. Clin Exp Metastasis, 1992. 10(2): p. 111-20.
17. Guan, J. L., Focal adhesion kinase in integrin signaling. Matrix Biol, 1997. 16(4): p. 195-200.
18. Chamber B A, L. D., Cancer chemotherapy and biotherapy. 2001: Lippincott, Williams and Wilkins.
19. Molhoek, K. R., et al., Human melanoma cytolysis by combined inhibition of mammalian target of rapamycin and vascular endothelial growth factor/vascular endothelial growth factor receptor-2. Cancer Res, 2008. 68(11): p. 4392-7.
20. Andarawewa, K. L., et al., Ionizing radiation predisposes nonmalignant human mammary epithelial cells to undergo transforming growth factor beta induced epithelial to mesenchymal transition. Cancer Res, 2007, 67(18): p. 8662-70.
21. Hong et al., 2010, J. Surgical Oncology, 101:564-569, ALCAM is associated with chemoresistance and tumor cell adhesion in pancreatic cancer.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

What is claimed is:

1. A method for enhancing sensitivity of a melanoma cell to a chemotherapeutic agent wherein said cell is therapy resistant, said method comprising contacting said cell with an effective amount of at least one integrin signaling stimulating agent and optionally at least one inhibitor of ALCAM levels or activity, thereby enhancing sensitivity of said melanoma cell to a chemotherapeutic agent.

2. The method of claim 1, wherein said integrin signaling stimulating agent is a multimeric fibronectin construct comprising at least two fibronectin peptides and a linker.

3. The method of claim 2, wherein said multimeric fibronectin construct comprises the linker short cartilage oligomeric peptide (COMP) or a homolog or fragment thereof.

4. The method of claim 3, wherein said multimeric fibronectin construct comprises five fibronectin peptides linked by COMP or a homolog or fragment thereof.

5. The method of claim 2, where each of said fibronectin peptides comprises at least one type III repeat.

6. The method of claim 5, wherein each of said fibronectin peptides comprises at least two type III repeats.

7. The method of claim 6, wherein each of said fibronectin peptides comprises type III repeats 5, 6, 7, 8, 9, 10, and 11.

8. The method of claim 3, wherein said COMP comprises the assembly domain of COMP.

9. The method of claim 8, wherein said assembly domain comprises amino acid residues 27-84 of COMP.

10. The method of claim 3, wherein said linker comprises amino acid residues 27-84 of COMP, said construct is a homo-multimeric construct comprising five fibronectin peptides, wherein each of said fibronectin peptides comprises type III repeats 5, 6, 7, 8, 9, 10, and 11 of fibronectin (FN-COMP).

11. The method of claim 1, wherein said integrin signaling agent binds to at least one integrin on said melanoma cell.

12. The method of claim 11, wherein said at least one integrin is selected from the group consisting of α5β1, αVβ3, and α4β1.

13. The method of claim 1, further wherein said cell is contacted with an effective amount of an inhibitor of ALCAM levels or activity.

14. The method of claim 1, wherein said integrin signaling stimulating agent is FN-COMP.

15. The method of claim 13, wherein said inhibitor is an siRNA directed against ALCAM.

16. The method of claim 15, wherein said siRNA has the sequence of SEQ ID NO:4 or SEQ ID NO:5.

17. The method of claim 13, wherein said inhibitor of ALCAM levels or activity is an antibody directed against ALCAM.

18. The method of claim 17, wherein said antibody is a monoclonal antibody, AZN-L50.

19. The method of claim 18, wherein said monoclonal antibody is AZN-L50.

20. The method of claim 1, wherein said chemotherapeutic agent is selected from the group consisting of dacarbazine, temozolomide, taxanes, nab-paclitaxel, paclitaxel, nitrosureas, carmustine, platinum-based agents, cisplatin, carboplatin, oxaliplatin, satraplatin, picoplatin, nedaplatin, triplatin, lipoplatin, and vinblastine.

21. A multimeric fibronectin construct for stimulating integrin signaling, said construct comprising at least two fibronectin peptides and a linker.

22. The multimeric fibronectin construct of claim 21, wherein said multimer is a homo-multimer of fibronectin peptides.

23. The multimeric fibronectin construct of claim 21, wherein said construct comprises five fibronectin peptides.

24. The multimeric fibronectin construct of claim 21, wherein said construct is FN-COMP.

25. The multimeric fibronectin construct of claim 21, wherein said multimeric fibronectin construct is a homo-multimer or heteromultimer of fibronectin peptides.

26. The multimeric fibronectin construct of claim 21, wherein said multimeric fibronectin construct comprises the linker COMP or a fragment thereof.

27. The multimeric fibronectin construct of claim 26, wherein said multimeric fibronectin construct comprises five fibronectin peptides linked by COMP or a homolog or fragment thereof.

28. The method of claim 25, wherein each of said fibronectin peptides comprises at least one type III repeat of fibronectin.

29. The multimeric fibronectin construct of 28, wherein each of said fibronectin peptides comprises at least two type III repeats.

30. The multimeric fibronectin construct of claim 29, wherein each of said fibronectin peptides comprises type III repeats 5, 6, 7, 8, 9, 10, and 11.

31. The multimeric fibronectin construct of claim 26, wherein said COMP fragment comprises the assembly domain of COMP.

32. The multimeric fibronectin construct of claim 31, wherein said assembly domain comprises amino acid residues 27-84 of COMP.

33. The multimeric fibronectin construct of claim 26, wherein said linker comprises amino acid residues 27-84 of COMP, said construct is a homo-multimeric construct comprising five fibronectin peptides, wherein each of said fibronectin peptides comprises type III repeats 5, 6, 7, 8, 9, 10, and 11 of fibronectin (FN-COMP).

34. The multimeric fibronectin construct of claim 21, wherein said construct binds to at least one integrin on said melanoma cell.

35. The multimeric fibronectin construct of claim 34, wherein said at least one integrin is selected from the group consisting of α5β1, αVβ3, and α4β1.

36. The method of claim 21, wherein said fibronectin has the sequence of SEQ ID NO:1, or a fragment or homolog thereof.

37. A method for treating a melanoma having detachment induced-chemoresistance wherein said method enhances sensitivity of said melanoma to a chemotherapeutic agent, said method comprising administering to a subject in need thereof a pharmaceutical composition comprising an effective amount of a chemotherapeutic agent and at least one of an effective amount of at least one integrin signaling stimulating agent or an effective amount of at least one inhibitor of ALCAM levels or activity, and optionally an additional therapeutic agent.

38. The method of claim 37, wherein said integrin signaling stimulating agent is a multimeric fibronectin construct comprising at least two fibronectin peptides and a linker.

39. The method of claim 38, wherein said multimeric fibronectin construct comprises the linker short cartilage oligomeric peptide (COMP) or a fragment thereof.

40. The method of claim 39, wherein said multimeric fibronectin construct comprises five fibronectin peptides linked by COMP.

41. The method of claim 38, where each of said fibronectin peptides comprises at least one type III repeat.

42. The method of claim 41, wherein each of said fibronectin peptides comprises at least two type III repeats.

43. The method of claim 42, wherein each of said fibronectin peptides comprises type III repeats 5, 6, 7, 8, 9, 10, and 11.

44. The method of claim 39, wherein said COMP comprises the assembly domain of COMP.

45. The method of claim 44, wherein said assembly domain comprises amino acid residues 27-84 of COMP.

46. The method of claim 39, wherein said linker comprises amino acid residues 27-84 of COMP, said construct is a homo-multimeric construct comprising five fibronectin peptides, wherein each of said fibronectin peptides comprises type III repeats 5, 6, 7, 8, 9, 10, and 11 of fibronectin (FN-COMP).

47. The method of claim 37, wherein said integrin signaling agent binds to at least one integrin on a melanoma cell of said melanoma.

48. The method of claim 47, wherein said at least one integrin is selected from the group consisting of α5β1, αVβ3, and α4β1.

49. The method of claim 37, further wherein an effective amount of an inhibitor of ALCAM levels or activity is administered to said subject.

50. The method of claim 37, wherein both an effective amount of said at least one integrin signaling stimulating agent and an effective amount of said at least one inhibitor of ALCAM levels or activity are administered to said subject.

51. The method of claim 37, wherein prior to said treatment it is determined whether said melanoma is a detachment-induced therapy resistant melanoma and a treatment is designed based on the outcome of the determination.

52. The method of claim 51, wherein it is determined whether said melanoma is a detachment-induced therapy resistant melanoma by measuring the level of ALCAM in said melanoma, comparing said level of ALCAM with the level of ALCAM in a sample from a second melanoma that is not detachment-induced therapy resistant or with a standard sample comprising a known level of ALCAM, wherein a higher level of ALCAM in said melanoma compared to said second melanoma or said standard is an indication that said melanoma is detachment-induced therapy resistant, thereby determining whether a melanoma is a detachment-induced therapy resistant melanoma.

53. The method of claim 37, wherein said inhibitor of ALCAM levels or activity is an antibody.

54. The method of claim 53, wherein said antibody is administered at a dosage from about 0.01 mg/kg to about 100 mg/kg, about 0.1 mg/kg to about 75 mg/kg, about 0.5 mg/kg to about 50 mg/kg, about 1.0 mg/kg to about 25 mg/kg, about 2.0 mg/kg to about 20 mg/kg, about 3.0 mg/kg to about 15 mg/kg, about 4.0 mg/kg to about 10 mg/kg, about 5.0 mg/kg to about 7.5 mg/kg, or as a unit dose.

55. The method of claim 37, wherein said integrating signaling stimulating agent is FN-COMP.

56. The method of claim 55, wherein said FN-COMP is administered at a dosage of about 0.1 mg/kg to about 100 mg/kg, about 1.0 mg/kg to about 75 mg/kg, about 5.0 mg/kg to about 50 mg/kg, about 10 mg/kg to about 25 mg/kg, about 15 mg/kg to about 20 mg/kg, or as a unit dose.

57. A method for determining whether a melanoma is a detachment-induced therapy resistant melanoma, said method comprising measuring the level of ALCAM in said melanoma, comparing said level of ALCAM with the level of ALCAM in a sample from a second melanoma that is not detachment-induced therapy resistant or with a standard sample comprising a known level of ALCAM, wherein a higher level of ALCAM in said melanoma compared to said second melanoma or said standard is an indication that said melanoma is detachment-induced therapy resistant, thereby determining whether a melanoma is a detachment-induced therapy resistant melanoma.

58. The method of claim 57, wherein a high level of ALCAM is an indication that said melanoma will be responsive to a treatment comprising integrin signaling stimulation in conjunction with chemotherapy.

59. The method of claim 57, wherein a treatment regimen is designed based on the ALCAM level of said melanoma.

60. The method of claim 59, wherein said treatment comprises administering to a subject in need thereof a pharmaceutical composition comprising a chemotherapeutic agent and at least one of an effective amount of at least one integrin signaling stimulating agent or an effective amount of at least one inhibitor of ALCAM levels or activity, and optionally an additional therapeutic agent.

61. The method of claim 57, wherein said ALCAM level measured is ALCAM protein level, ALCAM mRNA level, or both protein and mRNA levels.

62. A method for differentiating a detachment-induced therapy resistant melanoma from a therapy sensitive melanoma, said method comprising measuring the level of ALCAM in a test melanoma, comparing said level of ALCAM with the level of ALCAM in a sample from a second melanoma that is not detachment-induced therapy resistant or with a standard sample comprising a known level of ALCAM, wherein a higher level of ALCAM in said test melanoma compared to said second melanoma or said standard is an indication that said melanoma is detachment-induced therapy resistant, thereby differentiating a detachment-induced therapy resistant melanoma from a therapy sensitive melanoma.

63. A method for treating chemoresistant melanoma in a subject in need thereof wherein said chemoresistance is associated with high ALCAM levels in said melanoma, said method comprising administering to said subject a pharmaceutical composition comprising a pharmaceutically acceptable carrier and an effective of amount of at least one chemotherapeutic agent, an effective amount of at least one integrin signaling stimulator, optionally at least one inhibitor of ALCAM levels or activity, and optionally an additional therapeutic agent, thereby treating chemoresistant melanoma in a subject in need thereof.

64. A method for establishing a treatment regimen for a subject with melanoma, said method comprising determining the level of ALCAM expression in said melanoma, comparing said level of ALCAM with the level of ALCAM in a sample from a second melanoma that is not detachment-induced therapy resistant or with a standard sample comprising a known level of ALCAM:
a) wherein a higher level of ALCAM in said test melanoma compared to said second melanoma or said standard is an indication that said melanoma is detachment-induced therapy resistant, thereby differentiating a detachment-induced therapy resistant melanoma from a therapy sensitive melanoma, and establishing a treatment plan for said subject comprising administering a pharmaceutical composition comprising an effective amount of at least one chemotherapeutic or other therapeutic agent, an effective amount of at least one integrin stimulating agent, and optionally at least one inhibitor of ALCAM levels or activity; or
b) wherein a similar level of ALCAM in said test melanoma compared to said second melanoma that is not detachment-induced therapy resistant or compared to a standard sample comprising a known level of ALCAM is an indication that said test melanoma is not detachment-induced therapy resistant and establishing a treatment plan for said subject comprising administering a pharmaceutical composition comprising an effective amount of a standard melanoma chemotherapeutic agent or other therapy or agent.

65. The method of claim 64, wherein said method is useful for predicting responsiveness to chemotherapy.
